SEQUENCE LISTING (13 sequences)

SEQ ID NO: 1 - DNA, 20 bp, synthetic construct
  source 1..20: mol_type = other DNA
ccttcgcaactgcgttacac

SEQ ID NO: 2 - DNA, 20 bp, synthetic construct
  source 1..20: mol_type = other DNA
cactcaggactcatccgaac

SEQ ID NO: 3 - DNA, 20 bp, synthetic construct
  source 1..20: mol_type = other DNA
cgtgtgagtcccggagtaac

SEQ ID NO: 4 - DNA, 20 bp, synthetic construct
  source 1..20: mol_type = other DNA
cccggttaacgaccaacaac

SEQ ID NO: 5 - DNA, 20 bp, synthetic construct
  source 1..20: mol_type = other DNA
ccggaacttcaactgtggac

SEQ ID NO: 6 - DNA, 20 bp, synthetic construct
  source 1..20: mol_type = other DNA
cgccttgttccctcttctac

SEQ ID NO: 7 - DNA, 20 bp, synthetic construct
  source 1..20: mol_type = other DNA
cctagaagccgttggaaccc

SEQ ID NO: 8 - DNA, 20 bp, synthetic construct
  source 1..20: mol_type = other DNA
caagtgccacacaatagccc

SEQ ID NO: 9 - DNA, 19 bp, synthetic construct
  source 1..19: mol_type = other DNA
cacgacgctcttccgatct

SEQ ID NO: 10 - DNA, 20 bp, synthetic construct
  source 1..20: mol_type = other DNA
agatcggaagagcacacgtc

SEQ ID NO: 11 - DNA, 20 bp, synthetic construct
  source 1..20: mol_type = other DNA
agatcggaagagcacacgtc

SEQ ID NO: 12 - DNA, 58 bp, synthetic construct
  source 1..58: mol_type = other DNA
aatgatacggcgaccaccgagatctacactctttccctacacgacgctcttccgatct

SEQ ID NO: 13 - DNA, 64 bp, synthetic construct
  source 1..64: mol_type = other DNA
caagcagaagacggcatacgagatcgtgatgtgactggagttcagacgtgtgctcttccgatct

(19) United States
(12) Patent Application Publication
(10) Pub. No.: US 2026/0109968 A1
(43) Pub. Date: Apr. 23, 2026

(54) A METHOD FOR SIMULTANEOUS SYNTHESIS OF A PLURALITY OF OLIGONUCLEOTIDES

(71) Applicant: ELEVEN THERAPEUTICS LTD, Ramat Gan (IL)

(72) Inventors: Omer ZIV, Cambridge (GB); Cameron THORPE, Weymouth (GB); Arun TANPURE, Cambridge (GB); Yaniv ERLICH, Raanana (IL)

(21) Appl. No.: 19/105,703

(22) PCT Filed: Aug. 31, 2023
(86) PCT No.: PCT/EP2023/073932

§ 371 Date: Feb. 21, 2025

(30) Foreign Application Priority Data
Sep. 2, 2022 (EP) 22193795.6
Sep. 20, 2022 (EP) 22196662.5

(51) Int. Cl. C12N 15/10; C40B 40/06; C40B 50/14

(52) U.S. Cl. CPC C12N 15/1065; C12N 15/1068; C40B 40/06; C40B 50/14

(57) ABSTRACT

Disclosed is a method and compounds useful for performing said method for simultaneous synthesis of a plurality of oligonucleotide molecules, and more specifically for orthogonal synthesis of at least two different oligonucleotide molecules at the same time with applications in combinatorial chemistry and DNA encoded libraries (DEL).

### CROSS-REFERENCE TO RELATED APPLICATIONS

This application is the National Stage entry of International Application No. PCT/EP2023/073932, filed Aug. 31, 2023, which claims priority to EP patent application Ser. No. 22/193,795.6, filed Sep. 2, 2022, and EP patent application Ser. No. 22/196,662.5, filed Sep. 20, 2022.

### REFERENCE TO SEQUENCE LISTING SUBMITTED AS A COMPLIANT XML FILE (.xml)

A Sequence Listing is submitted herewith as an XML file named “20250211 P 1079 WO SEQUENCE LISTING 2.xml” created on Feb. 20, 2025, and having a size of 12,376 bytes as permitted under 37 C.F.R. § 1.821 (c). The material in the aforementioned file is hereby incorporated by reference in its entirety.

### BACKGROUND

### Field

The present invention relates to a method for simultaneous synthesis of oligonucleotides molecules, and more specifically for orthogonal synthesis of at least two chemically different nucleic acid molecules on a single solid support unit useful, for instance, for applications in combinatorial chemistry and DNA encoded libraries (DEL).

### Description of Related Art

Chemical synthesis of oligonucleotides is a cyclic process in which monomers are added in a stepwise manner to create the molecule of interest. The process relies on a basic cycle in which the nascent molecules are protected, blocking any attempt to add any new monomer. Next, the molecules are subject to a deblocking step that selectively removes the protective group and exposes a moiety that is amenable for extension. Finally, a protected monomer of choice is added, growing the molecule by another building unit. The process is repeated until the molecule of interest is completed. This process can have certain optional variations such as adding a capping step to block any extension of molecules that failed to incorporate a monomer or additional auxiliary steps such as oxidation to ensure that proper bonds are formed.

While multiple oligonucleotide synthesis schemes have been devised, the Caruthers method, which was developed over 25 years ago, is still the most reliable and proliferated strategy. This strategy is based on phosphoramidite monomers whose 5′ end is protected with DMTr (4,4′-dimethoxytrityl). De-blocking is achieved by washing the synthesis column with acid such as 3% trichloroacetic acid (TCA). Typical results of this scheme with DNA obtain a coupling efficiency of 99.5% per cycle.

The coupling efficiency is exponentially important as the synthesis length increases. Let x denote the coupling efficiency and n the length of the oligo. Then, the fraction of a molecule without coupling failures scales with xⁿ. For instance, a synthesis process would yield 36% molecules without any coupling failure when the coupling efficiency is 99%. The same process would yield 99% molecules without a coupling failure when the coupling efficiency is 99.99%, a mere 1% difference. Thus, optimizing the chemistry scheme is of utmost importance, specifically for biomedical applications.

Simultaneous synthesis of a plurality, more particularly two or more distinct oligonucleotide molecules have multiple applications.

One particular case of interest is DNA-encoded libraries (DEL) for oligonucleotide therapeutics (DEL-ON). DEL is a concept in which potential chemical entities are generated in a series of combinatorial split-pool reactions. In the first round of the split-pool, there are multiple reaction vessels. In each vessel, a building block is attached to a DNA barcode whose sequence signifies the identity of the reaction vessel. Next, all the vessels are pooled. The process repeats by splitting again the molecules from the first cycle in multiple reaction vessels, adding a different building block in each vessel and attaching again a DNA barcode to signify the reaction vessel. After multiple split-pool rounds, it is possible to generate an exponential number of chemical entities, each of which is labeled by a DNA molecule that signifies the identity of the reaction vessels that produced the chemical entity. The DNA molecule can be sequenced in order to reveal the identity of the chemical entity.

In the case of DEL-ON, the chemical entity is a biological-active oligonucleotide, such as an ASO, siRNA, aptamer, mRNA, LNA, gRNA for ADAR2 reprogramming, or gRNA for CRISPR/Cas9, CRISPR/Cas13, or CRISPR/Cas7/11. In these cases, it is desired to create a combinatorial library that varies these biological-active oligonucleotides. Examples of variations include modifications to the ribose such as 2′-F or 2′-O-methyl; modifications to the phosphate backbone such as introducing phosphonothioate links, 3′-phosphonothioate links, or 5′-phosphonothiolate links; or modifications to the nucleobases such as N1-methyl-pseudo-uridine, pseudo-uridine, or 2,6-diaminopurine.

Another application of simultaneous synthesis of two or more distinct oligonucleotides is the synthesis of double stranded oligonucleotides in one reaction. For example, in the case of siRNA, the simultaneous synthesis of two distinct oligonucleotide molecules can generate the guide and the passenger strand in one reaction vessel. This is of particular importance when considering combinatorial variations to the siRNA molecule. For example, when varying the guide strand, it is desired to vary the reverse complement nucleotide on the passenger strand. The simultaneous synthesis of two distinct oligonucleotide molecules will accomplish this task and ensure that the two corresponding nucleotides on the double strand molecule will reverse complement each other.

Another application of simultaneous synthesis of two or more distinct oligonucleotides can be DNA origami. This concept refers to building 2D and 3D shapes out of DNA molecules. The DNA molecules exhibit carefully designed complementarities between pairs of molecules that facilitate the formation of the structures. Simultaneous synthesis will allow to efficiently create in one reaction all the necessary molecules to create part of the entire structure.

Regardless of the application, the main challenge is establishing a reliable synthesis process that creates at least two types of molecules while maintaining high efficacy and minimal cross talk between the synthesis processes.

One potential solution for simultaneous synthesis is to use nucleotides with orthogonal protective groups on their 5′ end. For instance, in the case of simultaneous synthesis of two oligonucleotides, one can consider using the acid-labile DMTr protective group and a base-labile protective group such as 9-fluorenylmethoxycarbonyl (Fmoc). Another option is to use the DMTr and a UV-labile protective group such as 2-(2-nitrophenyl) propoxycarbonyl (NPPOC).

For instance, Gaytán et al. (Nucleic Acid Research, 2001; PCMID: PMC2764442) developed a process of simultaneous synthesis of a plurality of open reading frames by randomly incorporating DMTr-protected trimers or Fmoc dimers. The Fmoc dimers were selectively deprotected and were followed by a single addition of a DMTr-protected nucleosides to complete a codon. This method was reported to support variability of 5 loci along the oligonucleotides. In another example, de-Rochambeau (PHD thesis, University of McGill, 2019) reported the synthesis of DNA-Encoded functionalized aptamers, which consist of a DNA barcode and an RNA aptamer molecule. This method used the levulinyl (Lev) protected nucleosides for selective incorporation events to the DNA barcode and DMTr-protected nucleosides for synthesizing the RNA aptamer. This method supported only variability in seven loci across the RNA aptamer.

The one-protective group for each oligonucleotide type above has multiple drawbacks. First, only one molecule can utilize the highly efficient, commodity-scale DMTr chemistry. Synthesizing other molecules would necessitate utilizing protective groups that have been less optimized for nucleotide monomers, are far more expensive, and require custom synthesis in many cases. Second, even minimal cross talk between the chemistries can further reduce the efficacy of the synthesis process when repeated multiple times. Finally, the risk of cross talk increases as a function of the number of oligonucleotide types and also the costs of deploying all sorts of nucleoside monomers with less standard protective groups.

Therefore, the objective of the present invention was to provide a method allowing the simultaneous synthesis of two or more distinct oligonucleotide molecules at the same reaction vessel while maximizing the usage of the one optimized protective group and optionally deploying a split-pool process.

### SUMMARY

The present invention relates to a method for simultaneous synthesis of a plurality of oligonucleotides and compounds useful for such methods.

A first aspect of the present invention relates to a method for simultaneous synthesis of a plurality of oligonucleotides, comprising or consisting of the following steps:
(a) providing solid support particles comprising at least two reactive moieties, wherein one reactive moiety is accessible for chemical coupling of a building block and one moiety is blocked by a first protective group, a first stage building block and a second stage building block;
(b) coupling said first stage building block to said accessible reactive moiety generating a solid support intermediate;
(c) coupling a second protective group to said coupled first stage building block of the solid support intermediate thus obtained;
(d) removing the first protective group of the solid support intermediate thus obtained allowing access to the reactive moiety;
(e) coupling a second building block to said accessible reactive moiety thus obtained;
(f) optionally coupling the reactive moiety of the support intermediate thus obtained with protective group x, removing said second protective group, and repeating steps (b) to (e) for n times, wherein the first and second building block can differ from the building blocks provided in the previous cycle;
wherein n denotes an integer number of at least 1,
the second protective group is orthogonal to the first protective group and protective group x is orthogonal to the second protective group.

In some embodiments said solid support particles of step (a) comprise a plurality of reactive moieties, wherein at least one reactive moiety is accessible for chemical coupling of a building block and one moiety is blocked by a first protective group. In some embodiments said solid support unit is selected from the group of glass, poly(acryloylmorpholine), silica, controlled pore glass (CPG), polystyrene, polystyrene/latex, teflon, acrylate, polyacrylamide, agar, agarose, chemically modified agars and agaroses, carboxyl modified teflon, nylon and nitrocellulose. In some embodiments, said solid support further comprises an asymmetric doubler.

In some embodiments the first and/or second stage building block comprises or consists of a designated protective group. In some embodiments the first protective group, second protective group, and the designated protective group are chemically orthogonal to each other. In some embodiments steps (b) and (e) comprise at least one reaction cycle comprising coupling and deprotection of the building blocks, generating a solid support intermediate.

In some embodiments the building blocks and/or sub-building blocks utilize phosphoramidite chemistry. In a preferred embodiment, said building block and/or sub-building block is selected from the group of nucleotides, oligonucleotides, nucleosides and/or deoxynucleosides. In a more preferred embodiment, the building block is selected from table 1.

In some embodiments the first and/or second stage building block is coupled to the accessible reactive moiety using a designated group. In some embodiments the protective groups of the method according to the invention are independently selected from Table 2. In a preferred embodiment, the designated protective group is selected from the group of DMTr, Levulinyl and Fmoc. In an even more preferred embodiment, said designated protective group is DMTr. In some embodiments the first protective group and second protective group are either Levulinyl or Fmoc, and the first protective group and the second protective group are different from each other. In some embodiments the protective group x is the same as the first protective group.

In some embodiments the first and/or second building block is assembled by coupling sub building blocks to the respective accessible moiety using the designated group until the chemical structure of the complete building block has been assembled.

In some embodiments the method according to the invention further comprises step (g) releasing the plurality of oligonucleotides thus obtained from the solid support particle.

In some embodiments said at least one oligonucleotide of said plurality of oligonucleotides comprises a barcode allowing the identification of the chemical identity of the other oligonucleotides coupled to the same solid support particle.

In some embodiments steps (b) to (f) are performed during at least one round of split-pool synthesis.

A further aspect of the present invention relates to a compound according to formula (I)

$${( L 1 )}_{x} - T - {( L 2 )}_{y} \qquad formula ( I )$$

wherein
T denotes one of the following items: a solid support particle, a brancher, or a doubler, or a solid support particle connected to an asymmetric doubler or brancher;

### L1 Denotes a Spacer of Formula (Ia):

$$- {( S 1 )}_{n} - {( B 1 )}_{m} - P 1 \qquad formula ( Ia )$$

### L2 Denotes a Spacer of Formula (Ib):

$$- {( S 2 )}_{o} - {( B 2 )}_{p} - {( P 2 )}_{q} \qquad formula ( Ib )$$

S1 and S2 independently from each other denote a linker,
B1 and B2 independently from each other denote a building block,
P1 denotes a protective group,
P2 denotes a second protective group orthogonal to P1,
n, m, o, and p independently from each other denote 0 or an integer of at least 1,
x, y independently from each other denote an integer of at least 1, and
q denotes either 0 or 1.

In some embodiments P1 and P2 independently from each other are Fmoc or DMTr, while P1 and P2 are different from each other. In some embodiments, P1 and P2 independently from each other are Fmoc or Lev, while P1 and P2 are different from each other. In some embodiments, P1 and P2 independently from each other are Lev or DMTr, while P1 and P2 are different from each other.

In some embodiments T is a controlled pore glass bead (CPG). In some embodiments said CPG beads have pores of the size 1000 Å to 3000 Å. In some embodiments, the CPG beads are covered with an asymmetric doubler and/or branch. In some embodiments said doubler and/or brancher is 1-[5-(4,4′-dimethoxytrityloxy) pentylamide]-3-[5-levulinyloxypentylamido]-propyl-2-[(2-cyanoethyl)-(N,N-diisopropyl)]. In some preferred embodiments, T is branchers, doubler or dendrimer synthons covalently connected to a controlled pore glass or any other solid support.

### Definitions

For convenience, certain terms employed in the specification, examples, and appended claims are collected here.

As used herein, the singular form “a,” “an,” and “the” include plural references unless the context clearly dictates otherwise. For example, the term “target cell” also includes a plurality of target cells.

The terms “library” or “plurality” refer to a set of compounds or materials. A “combinatorial” library refers to a set of compounds or materials prepared by combinatorial chemistry. A library optionally includes a collection of pools or sub libraries. A library “member” refers, e.g., to a specific material or compound that is included in a library, or an uncharacterized physical product or material of a library synthesis. A member can refer to one or multiple molecules of the same structure. “Selected members” means a sub-set of compounds or materials, e.g., a collection of materials or compounds obtained from solid phase synthesis particles optionally with desired biological properties. A “selection of members” refers to a subgroup of individual members of the library, wherein the subgroup can comprise all members of the library or at least one member.

The term “solid support particle” refers to an insoluble, functionalized, polymeric material or particle to which library members or reagents may be attached (e.g., via a linker) allowing them to be readily separated (by filtration, centrifugation, etc.) from excess reagents, soluble reaction by-products or solvents. In the context of this invention, solid support particles can encompass the presence or absence of doubler, brancher, or a combination thereof.

The term “building block” refers to one of a number of interchangeable reagents which can be incorporated into a chemical polymer, such as DNA or RNA oligonucleotides. Building blocks are optionally used in combinatorial library synthesis, at least part of the structure of which becomes incorporated into an intermediate or final product such as a member of the plurality of oligonucleotides according to the invention. Building blocks may include a set of reagents that introduces diversity into library products and/or one that results in an identical conversion for each member of the library. Non-limiting examples of building blocks include nucleosides, chemically modified nucleosides, photo-labile linkers, fatty acid chains, natural and artificial amino-acids, amino-nucleotides, inverted nucleosides, 5′-capping reagents.

The term “linker” refers to a bifunctional chemical moiety attaching a compound to, e.g., a solid support which can be cleaved to release materials or compounds from the support. A careful choice of linker allows cleavage to be performed under appropriate conditions compatible with the stability of the compound and assay method. Additional description of linker molecules is provided in, e.g., Backes and Ellman (1997) “Solid support linker strategies,” Curr. Opin. Chem. Biol. 1:86-93, Backes et al. (1996)” Activation method to prepare a highly reactive acylsulfonamide “safety-catch” linker for solid-phase synthesis, “J. Amer. Chem. Soc. 118:3055-3056, Backes and Ellman (1994)” Carbon-carbon bond-forming methods on solid support. Utilization of Kenner's″Safety-Catch “linker,” J. Amer. Chem. Soc. 116:11171-11172, Hoffmann and Frank (1994) “A new safety-catch peptide-resin linkage for the direct release of peptides into aqueous buffers, “Tetrahedron Lett. 35:7763-7766, Kocis et al. (1993)” Symmetrical structure allowing the selective multiple release of a defined quantity of peptide from a single bead of polymeric support,” Tetrahedron Lett. 34:7251-7252, and Plunkett and Ellman (1995) “A silicon-based linker for traceless solid-phase synthesis,” J. Org. Chem. 60:6006-6007.

The term “cleavage” refers to a process of releasing a material or compound from e.g. a solid support particle, e.g., to permit analysis of the compound by sequencing. Cleaving can be by chemical, optical (e.g., UV-labile), and/or enzymatic reactions.

The term “split-pool synthesis” refers to a procedure in which the products of a plurality of first reactions are combined (pooled) and then separated (split) before participating in a plurality of second reactions. In general, the approach may be used for a variety of coupling reactions and conjugation chemistries including, but not limited to, amino acid (or short peptide) coupling reactions to produce longer peptides of fully or partially random amino acid sequences, the coupling of deoxyribonucleotides (or short DNA oligonucleotides) to produce longer DNA oligonucleotides of fully or partially random base sequences, or the coupling of ribonucleotides (or short RNA oligonucleotides) to produce longer RNA oligonucleotides of fully or partially random base sequences. Any of a variety of chemical monomers, e.g., amino acids, small molecules, short peptides, short oligonucleotides, etc., may thus be used as building blocks for assembly of unique barcodes.

The term “identify” refers to the identification of the structure of members of the compound library. “Identify” can further relate to identification of compound library members followed by production of individual said members by synthesis known in the art.

The term “structural identification” refers to the identification of all, or a constituent part (e.g., a substituent or functional group) of a compound's chemical or physical structure.

A “scaffold” or “template” refers to a core portion of a molecule common to all members of a combinatorial library or sub-library.

The terms “oligonucleotide” and “nucleic acid” are used herein interchangeably. They refer to a polymeric form of nucleotides of any length: Oligonucleotides may have any three-dimensional structure, and may perform any function, known or unknown. The following are non-limiting examples of oligonucleotides: coding or non-coding regions of a gene or gene fragment, exons, introns, messenger RNA (mRNA), transfer RNA, ribosomal RNA, ribozymes, cDNA, synthetic polynucleotides, recombinant polynucleotides, branched polynucleotides, plasmids, vectors, isolated DNA of any sequence, isolated RNA of any sequence, nucleic acid probes, and primers. An oligonucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. An oligonucleotide may be further modified, such as by conjugation with a labeling component.

If not stated otherwise, the term “nucleic acid” refers to any nucleic acid such as ribonucleic acid, deoxyribonucleic acid, xeno nucleic acid, single stranded or double stranded.

As used herein, the term “xeno nucleic acids” or “XNAs” are synthetic nucleic acid analogues that have a different phospho-sugar backbone or nucleobases than the natural nucleic acids DNA and RNA.

As used herein, two nucleic acid sequences “complement” one another or are “complementary” to one another if they base pair one another at each position. The term “complement” is defined as a sequence which pairs to a given sequence based upon the canonical base-pairing rules. For example, a sequence A-G-T in one nucleotide strand is “complementary” to T-C-A in the other strand The term “complement” and the phrase “reverse complement” are used interchangeably herein with respect to nucleic acids, and are meant to define the antisense nucleic acid.

As used herein, the term “barcode” or “sequence-controlled polymer” refers to a polymer sequence such as a nucleic acid sequence used to identify the sequence of split-pool events that generated the compound. The barcode can have error-detecting or error-correcting capabilities, such as Hamming codes. Oligonucleotides can serve as barcodes by themselves.

The terms “orthogonal chemistry” or “orthogonal protection” refers to protection strategy allowing the deprotection of functional groups independently of each other.

As used herein, the term “attachment” or “coupling” refers to the attachment of a reactive moiety of a reagent or building block to a reactive moiety of another building block or solid support particle creating a chemical bond such as an ionic bond, a covalent bond or a metallic bond. In a preferred embodiment “attached” refers to a covalent bond.

The terms “brancher” or a “doubler” are chemical molecules that have at least two attachment points that can be used to grow oligonucleotides. Typically, they also have an additional attachment point that can anchor them to a solid support particle. Unless otherwise stated, the doublers or branches are asymmetric, meaning that each attachment point for oligonucleotide synthesis has a distinct protective group. Non-limiting examples of such branches and doublers monomers include 1-[5-(4,4′-dimethoxytrityloxy) pentylamido]-3-[5-levulinyloxypentylamido]-propyl-2-[(2-cyanoethyl)-(N,N-diisopropyl)]-phosphoramidite or 5′-Dimethoxytrityl-N4-(O-levulinyl-6-oxyhexyl)-5-Methyl-2′-deoxyCytidine,3′-[(2-cyanoethyl)(N,N-diisopropyl)]-phosphoramidite or 1-[5-(4,4′-dimethoxytrityloxy) pentylamido]-3-[5-fluorenylmethoxycarbonyl]-propyl-2-[(2-cyanoethyl)-(N,N-diisopropyl)]-phosphoramidite.

Unless defined otherwise, the technical and scientific terms used herein have the same meaning as commonly understood by one of ordinary skill in the art to which the invention belongs. Particularly, unless otherwise stated, a term as used herein is given the definition as provided in the Oxford dictionary of biochemistry and molecular biology, Oxford University Press, 1997, revised 2000 and reprinted 2003, ISBN 0 19 850673 2.

### DETAILED DESCRIPTION OF A PREFERRED EMBODIMENT

A first aspect of the present invention relates to a method for the simultaneous synthesis of a plurality of oligonucleotides. The generation of multiple oligonucleotides that are chemically different in properties such as, for instance, base sequence, chemical modifications of nucleotides, and/or nucleic acid species in the same reaction vessel is critical for multiple applications such as barcoding strategies, wherein an oligonucleotide encodes the chemical identity of the other oligonucleotide in split-pool approaches, or the generation of complementary oligonucleotides able to form double stranded nucleic acids in a single reaction vessel.

Surprisingly, the method of the present invention allows the simultaneous synthesis of at least two different nucleic acid molecules in the same reaction vessels utilizing a solid support particle as scaffold for assembly of said oligonucleotides in combination with a synthesis approach, which maximizes the efficiency of a designated protective group for coupling individual building blocks such as nucleotides or oligonucleotides during the synthesis step. The ability to assemble building blocks iteratively in multiple cycles further allows the successive building of desired oligonucleotide combinations on the same solid support particle, which enables the efficient transfer of intermediates to different reaction vessels useful for split-pool approaches and thereby facilitates the generation of a plurality of oligonucleotides of distinctive chemical identity, wherein oligonucleotides attached to the same solid support particle can be easily identified for example by barcoding and sequencing.

The method according to the present invention comprises at least the steps (a) to (e). In a first step (a) a solid support particle is provided comprising at least two reactive moieties, wherein at least one reactive moiety is accessible for chemical coupling of a building block and at least one moiety is blocked by a first protective group. Additionally provided are a first stage building block and a second stage building block. In step (b) the first stage building block is coupled to the accessible reactive moiety thereby generating a solid support intermediate. In step (c) a second protective group is coupled to the coupled first stage building block of the solid support intermediate of step (b), wherein the second protective group is orthogonal to the first protective group. In step (d) the first protective group is removed thus revealing an accessible reactive moiety of the solid support intermediate. Step (e) comprises the coupling of a second building block to the accessible reactive moiety of step (d). In some embodiments the building blocks provided are nucleotides and/or oligonucleotides.

The combination of step (a) to (e) allows the generation of at least two oligonucleotides (first and second building block) attached to the same solid support particle. In some embodiments it may be necessary to further attach building blocks to the previous first and second building blocks. This allows the successive synthesis of longer oligonucleotides and the incorporation of various chemical modification patterns. Simultaneously the ability to generate two chemically distinct oligonucleotides on the same solid support particle enables barcoding strategies, wherein one oligonucleotide attached to the solid support particle encodes the chemical identity of the other oligonucleotide.

Accordingly, the method of the invention optionally comprises step (f), wherein the accessible reactive moiety of the support intermediate of the second building block is coupled with a protective group x, the second protective group of step (c) is removed thus revealing the accessible moiety of the solid support particle of the first stage building block, and steps (b) to (e) are repeated for at least n times, wherein the first and second building block can differ from the building blocks provided in the previous cycle, n denotes an integer of at least 1, and protective group x is orthogonal to the second protective group. In some embodiments n denotes an integer between 1 and 1000, preferably 3 and 100, more preferably 8 and 20.

One advantage of the method according to the invention is that the coupling of the building block of step (b) and (e) can be performed using a highly efficient strategy. While conventional synthesis of oligonucleotides using protective groups requires the use of said group for coupling reactions, thereby being highly dependent on the coupling efficiency of said selected protective group, the method according to the invention allows the selection of a designated protective group (=designated group) which can be used for coupling of the building blocks or sub building blocks. This allows the choice of highly effective protective groups such as DMTr for coupling reactions. Additionally, simultaneous synthesis of multiple distinct oligonucleotides on the same solid support particle utilizing protective groups orthogonal to each other and orthogonal to the designated group for protection of reactive moieties of the building block during the synthesis of the other oligonucleotide is possible.

### Solid Support Particles

The solid support matrix for oligonucleotide synthesis is designed to provide a stable and versatile platform for nucleotide coupling reactions. The matrix comprises a network of interconnected pores, with a typical pore size ranging from 50 to 300 nanometers, allowing for optimal diffusion of reagents and efficient utilization of reactive sites. The core of the solid support matrix is constructed from a chemically inert and mechanically stable material, such as cross-linked polystyrene, polyacrylamide, or silica gel which exhibits compatibility with standard oligonucleotide synthesis chemistry. The choice of core material ensures minimal interference with the synthesis reactions while providing a sturdy structure for attachment of functional groups. The surface of the core material is modified with specific functional groups that facilitate covalent attachment of nucleotide monomers during the synthesis process. These functional groups include amino, hydroxyl, or thiol moieties, which are either directly attached to the core material or introduced through spacer molecules to enhance accessibility and reactivity. In cases where spacer molecules are used, they are selected to provide appropriate distances between the core material and the reactive functional groups. This optimization ensures efficient reaction kinetics and minimizes steric hindrance during oligonucleotide elongation. To prevent undesirable side reactions and truncation products, the solid support matrix also incorporates capping groups that react with unreacted nucleotide monomers.

The solid support particles utilized in the methods of the invention include many alternative embodiments. For example, the solid support particles optionally each include a single particle independently selected from, e.g., a bead, a piece of polymer, or the like. Optionally, the solid support particles each include multiple particles combined together. For example, an array or a container optionally includes multiple particles combined together. In certain embodiments, at least one of the multiple particles includes a non-functionalized solid support, whereas in others, at least one of the multiple particles includes a solid support having one or more functionalities attached thereto. In some embodiments, at least two of the multiple particles include solid supports having one or more identical or different functionalities attached thereto.

Suitable solid support particle materials include, but are not limited to, glass, poly(acryloylmorpholine), silica, controlled pore glass (CPG), polystyrene, polystyrene/latex, teflon, acrylate, polyacrylamide, agar, agarose, chemically modified agars and agaroses, carboxyl modified teflon, nylon and nitrocellulose. The solid substrates can be biological, nonbiological, organic, inorganic, or a combination of any of these, existing as particles, strands, precipitates, gels, sheets, tubing, spheres, containers, capillaries, pads, slices, films, plates, slides, etc., depending upon the particular application. Other suitable solid substrate materials will be readily apparent to those of skill in the art.

Often, the surface of the solid substrate will contain reactive groups, such as carboxyl, amino, hydroxyl, thiol, or the like for the attachment of nucleic acids, proteins, etc. Surfaces on the solid substrate will sometimes, though not always, are composed of the same material as the substrate. Thus, the surface may be composed of any of a wide variety of materials, for example, polymers, plastics, resins, polysaccharides, silica or silica-based materials, carbon, metals, inorganic glasses, membranes, or any of the above-listed substrate materials. The surface may also be chemically modified or functionalized in such a way as to allow it to establish binding interactions with functional groups intrinsic to or specifically associated with the chemical materials to be immobilized.

In a preferred embodiment the solid support particles are made from materials selected from the group of silica gel, glass, resins, metal, polymers, and polysaccharide. In a preferred embodiment said material is selected from the group of porous glass beads, controlled pore glass beads (CPGs), polystyrene beads, and polyacrylamide beads. In a further preferred embodiment, the solid support particles are controlled pore glass beads (CPGs). In the most preferred embodiment, the CPG beads have a pore size of 1000 Å or 2000 Å.

### Reactive Moieties

Surface of the solid support matrix is functionalized with a range of chemically reactive groups. These reactive functionalities serve as sites for coupling and deprotection reactions during oligonucleotide synthesis. The invention provides the ability to incorporate multiple types of reactive groups, which are strategically placed in defined regions of the solid support matrix. The solid support matrix comprises distinct regions, each functionalized with a specific reactive group, enabling orthogonal and selective chemical reactions. The reactive functionalities include, but are not limited to Amino, Hydroxyl, Thiol, Carboxylic Acid, Photolabile Groups. The solid support matrix with multiple reactive functionalities offers flexibility in oligonucleotide synthesis, allowing for the creation of complex sequences. The distinct reactive groups enable orthogonal chemical reactions, reducing cross-reactions and enhancing sequence purity. The modular design of the solid support matrix allows for easy customization and adaptation to different oligonucleotide synthesis protocols.

The solid support particles provided according to the present invention may comprise a plurality of reactive moieties wherein at least one reactive moiety is accessible for chemical coupling of a building block and at least one moiety is blocked by a first protective group. In some embodiments, the solid support particles comprise at least two reactive moieties, wherein one reactive moiety is accessible for chemical coupling of a building block and one moiety is blocked by a first protective group. Reactive groups comprising said reactive moieties of the solid support particles may be present in a plurality of numbers on a single solid support particle. For instance, in some embodiments the solid support unit comprises at least two types of reactive moieties, an accessible reactive moiety and a protected reactive moiety, wherein each moiety is present on a single particle between 1 and about 10 to the power of 9 times. In a preferred embodiment, each moiety is present of the particle between about 100 and about 100,000,000 times, in a preferred embodiment, between about 1,000 and about 10,000,000 times, in a preferred embodiment, between about 10,000 and about 1,000,000 times, in a preferred embodiment between about 1,000 and about 100,000 times, in a preferred embodiment between about 100 and about 10,000 times. In some embodiments, the solid support is covered with a doubler or brancher with at least two types of the said reactive moieties, wherein one reactive moiety is accessible for chemical coupling of a building block and one moiety is blocked by a first protective group. In a preferred embodiment, the solid support is covered with an asymmetric doubler with exactly two reactive moieties, where one reactive moiety that is accessible for chemical coupling and other reactive moiety is protected by a protective group that is orthogonal to DMTr. In the most preferred embodiment, the orthogonal protective group is Lev or Fmoc.

Reactive moieties according to the present invention may be any reactive moiety known by the skilled person in the art useful for coupling nucleotides or oligonucleotides to said reactive moieties comprising the solid support particle. In some embodiments, the reactive moiety is selected from the group of carboxyl, amino, hydroxyl, and thiol. In more preferred embodiments, the reactive moiety is hydroxyl.

Reactive moieties accessible for chemical coupling (accessible reactive moieties) refers to unprotected reactive moieties (reactive moieties exposed to the environment and not attached to a protective group), which are able to undergo coupling reactions according to the present invention. Blocked reactive moieties refer to a reactive moiety, which is attached to a protective group as known by the skilled person. Blocked reactive moieties according to the invention may not be able to undergo coupling reactions, while the protective group is attached.

### Linkers and Linking Chemistry

The present invention describes linker and spacer designs for solid support oligonucleotide synthesis. The linkers and spacers provided herein enhance the efficiency of nucleotide coupling reactions, reduce side reactions, and improve the overall purity of synthesized oligonucleotides. The invention further offers versatility in choosing the appropriate linker and spacer combination based on the specific requirements of the oligonucleotide synthesis.

The invention comprises a spacer-embedded linker that connects the nucleotide to the solid support. The spacer-embedded linker includes a flexible oligomeric spacer, such as a polyethylene glycol (PEG) chain, which reduces steric hindrance during the coupling reaction. The linker is attached to the 3′-OH of the nucleotide or aminonucleoside and covalently binds to the solid support. The linker contains a reactive group, such as an alkyne or azide, enabling click chemistry reactions with complementary reactive groups on the solid support. This robust linkage prevents premature detachment of the nucleotide during synthesis and facilitates efficient coupling. In some embodiments, a photo-cleavable linker that allows controlled detachment of the oligonucleotide from the solid support is used. Upon completion of synthesis, exposure to a specific wavelength of light cleaves the linker, releasing the synthesized oligonucleotide. This feature enables easy purification and downstream applications. The invention encompasses a rigid spacer that minimizes conformational variability between nucleotides during synthesis. This spacer may include a short peptide chain or a rigid organic moiety, ensuring precise alignment of nucleotides and enhancing coupling efficiency. Variable length spacer composed of a series of repeating units. This design allows fine-tuning of the distance between the nucleotide and the solid support, optimizing coupling reactions for different nucleotide chemistries. The invention also provides a cleavable spacer that undergoes controlled cleavage after coupling, ensuring complete detachment of the nucleotide from the solid support while minimizing side reactions.

The reactive moieties comprising the solid support particles according to the invention may be linked via any of a variety of linkers to the particles, allowing the incorporation of biological and chemical components of interest into the solid support particles. A wide variety of organic and inorganic polymers, both natural and synthetic may be employed as the material for the solid surface. Illustrative polymers include a DNA or RNA sequence, including inosine as the last nucleotide of the linker. Other illustrative polymers include polyethylene, polypropylene, poly (4 methylbutene), polystyrene, polymethacrylate, poly(ethylene terephthalate), rayon, nylon, poly(vinyl butyrate), polyvinylidene difluoride (PVDF), silicones, polyformaldehyde, cellulose, cellulose acetate, nitrocellulose, and the like. Other materials that may be employed include galactose, or Valine-Alanine di-peptide, and UV sensitive materials, such as NPPOC, or photocaged photolinkers (photocleavable linkers), such as described in Zhang and Taylor (J. Am. Chem. Soc., 1999). In addition, substances that form gels, such as proteins (e.g., gelatins), lipopolysaccharides, silicates, agarose and are also optionally used.

A wide variety of linking chemistries are available for linking molecules to a wide variety of solid support particles. It is impractical and unnecessary to describe all the possible known linking chemistries for linking molecules to a solid support particle. It is expected that one of skill, can easily select appropriate chemistries, depending on the intended application. However, for purposes of illustration certain linkers and linkage chemistries are described. For example, in certain embodiments, solid support particles of the invention optionally include silicate elements (e.g., glass or silicate beads). Silicon-based molecules appropriate for functionalizing surfaces are commercially available.

Additionally, the art in this area is very well developed and those of skill will be able to choose an appropriate molecule for a given purpose. Appropriate molecules can be purchased commercially, synthesized de novo, or it can be formed by modifying an available molecule to produce one having the desired structure and/or characteristics.

The substrate linker attaches to the solid substrate through any of a variety of chemical bonds. For example, the linker is optionally attached to the solid substrate using carbon-carbon bonds, for example via substrates having (poly) trifluorochloroethylene surfaces, or siloxane bonds (using, for example, glass or silicon oxide as the solid substrate). Siloxane bonds with the surface of the substrate are formed in one embodiment via reactions of derivatization reagents bearing trichlorosilyl or trialkoxysilyl groups. The particular linking group is selected based upon, e.g., its hydrophilic/hydrophobic properties where presentation of an attached polymer in solution is desirable. Groups which are suitable for attachment to a linking group include amine, hydroxyl, thiol, carboxylic acid, ester, amide, isocyanate and isothiocyanate. Preferred derivatizing groups include aminoalkyltrialkoxysilanes, hydroxyalkyltrialkoxysilanes, polyethylene glycols, polyethyleneimine, polyacrylamide, polyvinylalcohol and combinations thereof.

The building blocks, which can be attached to a derivatized surface (reactive moieties), include for instance, but not limiting, peptides, nucleic acids, mimetics, large and small organic molecules, polymers, or the like. For example, moieties bearing a permanent charge or a pH dependent charge are useful in practicing the present invention. For example, the charged group can be a carboxylate, quaternary amine or protonated amine that is a component of an amino acid that has a charged or potentially charged side chain. The amino acids can be either those having a structure which occurs naturally or they can be of unnatural structure (i.e., synthetic). Useful naturally occurring amino acids include, arginine, lysine, aspartic acid and glutamic acid. Surfaces utilizing a combination of these amino acids are also of use in the present invention. Further, peptides comprising one or more residues having a charged or potentially charged side chain are useful coating components and they can be synthesized utilizing arginine, lysine, aspartic acid, glutamic acid and combinations thereof. Useful unnatural amino acids are commercially available or can be synthesized utilizing art-recognized methodologies.

In those embodiments in which an amino acid moiety having an acidic or basic side chain is used, these moieties can be attached to a surface bearing a reactive group through standard peptide synthesis methodologies or easily accessible variations thereof. See, e.g., Jones, Amino Acid and Peptide Synthesis, Oxford University Press, Oxford, 1992.

Linking groups can also be placed on the solid support particles of the invention. Linking groups of use in the present invention can have a range of structures, substituents and substitution patterns. They can, for example be derivatized with nitrogen, oxygen and/or sulfur containing groups which are pendent from, or integral to, the linker group backbone. Examples include, polyethers, polyacids (polyacrylic acid, polylactic acid), polyols (e.g., glycerol), polyamines (e.g., spermine, spermidine) and molecules having more than one nitrogen, oxygen and/or sulfur moiety (e.g., 1,3diamino-2-propanol, taurine). See, e.g., Sandler et al. Organic Functional Group Preparations, 2nd Ed., Academic Press, Inc. San Diego 1983. A wide range of mono-, di- and bis-functionalized poly (ethyleneglycol) molecules are commercially available and will prove generally useful in this aspect of the invention. See, e.g., 1997-1998 Catalog, Shearwater Polymers, Inc., Huntsville, Alabama. Additionally, those of skill in the art have available a great number of easily practiced, useful modification strategies within their synthetic arsenal. See, e.g., Harris, Rev. Macromol. Chem. Phys., C25 (3): 325-373 (1985); Zalipsky et al., Eur. Polym. J., 19 (12): 1177-1183 (1983); U.S. Pat. No. 5,122,614, issued Jun. 16, 1992 to Zalipsky; U.S. Pat. No. 5,650,234, issued to Dolence et al. Jul. 22, 1997, and references therein.

In some embodiments, the linker further comprises a polymer, wherein the reactive moiety of the solid support particle is positioned in said polymer. In some embodiments, said polymer is a peptide or oligonucleotide. In some embodiments, said polymer is an oligonucleotide comprising regulatory elements such as untranslated regions (UTRs), poly adenine tails or desired or random base sequences.

### Protective Groups and Protecting Chemistries

The present invention relates to methods for the efficient synthesis of oligonucleotides on solid supports, incorporating a variety of protective groups and protecting chemistries. The invention provides a versatile approach to oligonucleotide synthesis, allowing for the controlled manipulation of protective groups and protecting chemistries to enhance yield, purity, and overall efficiency. Various protective groups and protecting chemistries are employed to safeguard functional groups during synthesis, enabling the generation of diverse oligonucleotide sequences with high fidelity and reduced side reactions. An oligonucleotide synthesis method is disclosed that employs a combination of base- and phosphate-protective groups. Base-protective groups, such as benzoyl, isobutyryl, and acetyl, are strategically selected to prevent undesirable side reactions during deprotection and coupling steps. Phosphate-protective groups, such as cyanoethyl may be employed to shield the phosphate backbone, preventing undesired cross-coupling reactions. Wide range of 5′ and 2′ protective groups may be employed maintaining chemical orthogonality which allows the selective oligonucleotide elongation on desired chain.

Among a wide variety of protective groups which are useful in the scope of the invention are Dimethoxytrityl, [bis-(4-methoxyphenyl)phenylmethyl] (DMTr), nitroveratryl (NVOC)methylnitroveratryl (Menvoc), allyloxycarbonyl (ALLOC), fluorenylmethoxycarbonyl (FMOC),methylnitro-piperonyloxycarbonyl (MeNPOC), levulinyl, NH-FMOC groups, t-butyl esters, t-butyl ethers, and the like.

Various exemplary protective groups (including both photo-cleavable and non-photocleavable groups) are described in, e.g., Atherton et al., (1989) Solid Phase Peptide Synthesis, IRL Press, and Greene, et al. (1991) Protective Groups In Organic Chemistry. 2nd Ed., John Wiley & Sons, New York, NY, as well as, e.g., Fodor et al. (1991) Science, 251:767-777, Wang (1976) J. Org. Chem. 41:3258; and Rich, et al. (1975) J. Am. Chem. Soc. 97:1575 1579. The use of these and other photo-cleavable linking groups for nucleic acid and peptide synthesis on solid supports is a well-established methodology. Additional details relating to linkers and linkage chemistries is provided in, e.g., Alonso et al. (2000) “β-Dimethylphenylsilylethyl esters: A linker for solid phase chemistry,” Tetrahedron Lett. 41:5617-5622, Berst et al. (2000) “A latent aryl hydrazine' safety-catch' linker compatible with N-alkylation,” Tetrahedron Lett. 41:6649-6653, Blanco et al. (2000) “Solid phase Diels-Alder/retro Diels-Alder reactions. A new method for traceless linker strategy,” Tetrahedron Lett. 41:7875-7878, and Blaney et al. (2000) “Solid-phase synthesis of tertiary methylamines via reductive alkylation-fragmentation using a hydroxylamine linker,” Tetrahedron Lett. 41:66356638.

In certain embodiments of the invention, the protective groups chemistries for controlled coupling materials to the solid support particles of the invention are light-controllable, i.e., utilize photo-labile chemistries. Illustrative examples of such protective groups are 5′-(α-methyl-2-nitropiperonyl)oxycarbonyl (MeNPOC), dimethoxybenzoincarbonate (DMBOC), 2-(2-nitrophenyl) propoxycarbonyl (NPPOC), benzoyl-2-(2-nitrophenyl) propoxycarbonyl (BzNPPOC) and thiophenyl-2-(2-nitrophenyl) propoxycarbonyl (SPh-NPPOC). The use of photoreactive chemistries and masking strategies to activate coupling of molecules to substrates, as well as other photo reactive chemistries is generally known (e.g., for DNA microarray fabrication and for coupling bio-polymers to solid phase materials). The use of photo-cleavable protective groups and photo-masking permits type switching of both mobile and fixed array members, i.e., by altering the presence of substrates present on the array members (i.e., in response to light).

In a preferred embodiment of the method according to the invention, the designated group is selected from the group of DMTr, Fmoc and Levulinyl. In a more preferred embodiment, said designated group is DMTr.

While many combinations of orthogonal protective groups of designated group, first protective group, and second protective group are possible, in a preferred embodiment, the designated group is DMtr, the first and second protective group are either Fmoc or Levulinyl, wherein the first and second protective group are different from each other.

### Building Blocks

A building block refers to one of a number of interchangeable reagents which may be optionally used in combinatorial library synthesis. Building blocks may include a set of reagents that introduces diversity into library products and/or one that results in an identical conversion for each member of the library.

In a preferred embodiment, building blocks utilize phosphoramidite chemistry. In another preferred embodiment, building blocks are chemically unmodified or modified nucleosides or oligonucleotides. In a more preferred embodiment said nucleosides or oligonucleotides are phosphoramidite nucleosides or oligonucleotides. In some embodiments, the building blocks comprise at least one amino acid codon. In some embodiments said at least one amino acid codon comprises an identifying nucleotide at position 3 to create a silent mutation. In some embodiments, the building blocks further comprise regulatory regions such as 3′ or 5′ untranslated regions (UTRs), restriction enzyme recognition sites and PCR amplification sites. In some embodiment building blocks comprise oligonucleotides allowing the identification of the oligonucleotide structure based on the base sequence.

In some embodiments building blocks are A, G, C, T/U Phosphoramidites. These are the basic monomers corresponding to the four DNA (A, G, C, T) or RNA (A, G, C, U) bases. Diverse range of phosphoramidite monomers used in solid-phase oligonucleotide synthesis which consist of various 2′ protective groups are known in the art. Each monomer may have a phosphoramidite group protecting at 3′ while the 5′-hydroxyl contains either DMTr, Lev or Fmoc protective group.

In some embodiments the building blocks or sub building blocks according to the invention are selected from the group of compounds of formula A to L in table 1:

TABLE 1

| ID | Name | Formula |
|---|---|---|
| A | dT-CE Phosphoramidite (98796- 51-1) | 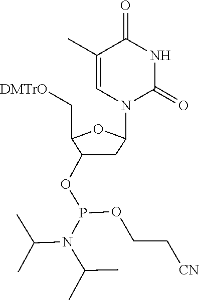 |
| B | dA-CE Phosphoramidite (98796-53-3) | 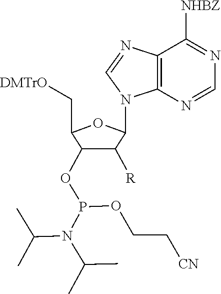 |
| C | dC-CE Phosphoramidite (102212-98-6) | 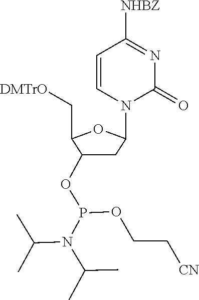 |
| D | dG-CE Phosphoramidite (93183-15-4) | 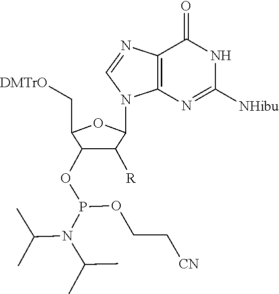 |
| E | 5-FMOC-dT 3′CE Phosphoramidite | 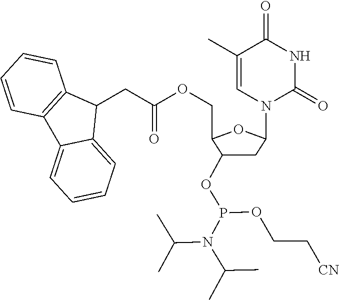 [SMILES: Cc1cn(C2CC(OP(OCCC#N)N(C(C)C)C(C)C)C(COC(=O)CC3c4ccccc4-c4ccccc43)O2)c(=O)[nH]c1=O] |
| F | 5′-FMOC-dG(n-ibu) 3′CE Phosphoramidite | 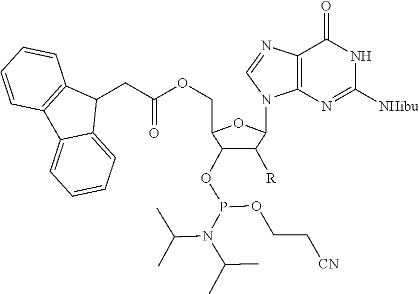 |
| G | 5′-FMOC-dC(n-Bz) 3′CE Phosphoramidite | 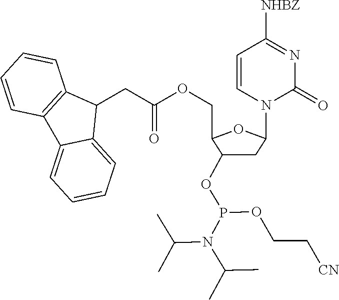 |
| H | 5′-FMOC-dA(n-Bz) 3′CE Phosphoramidite | 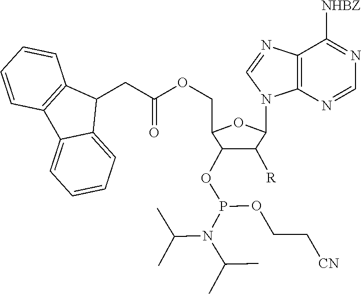 |
| I | 5′-Levulinyl-dT 3′CE Phosphoramidite | 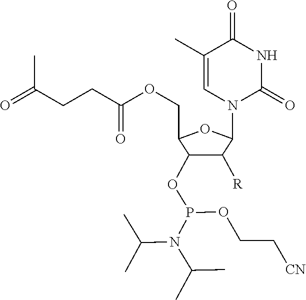 |
| J | 5′-Levulinyl-dG(n-ibu) 3′CE Phosphoramidite | 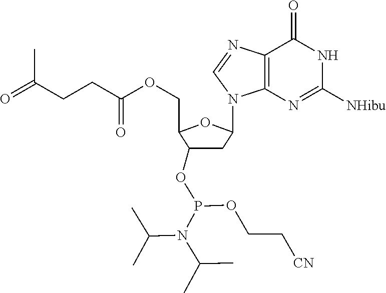 |
| K | 5′-Levulinyl-dC(n-Bz) 3′CE Phosphoramidite | 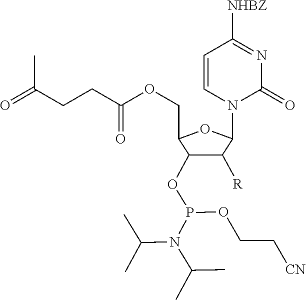 |
| L | 5′-Levulinyl-dA(n-Bz) 3′CE Phosphoramidite | 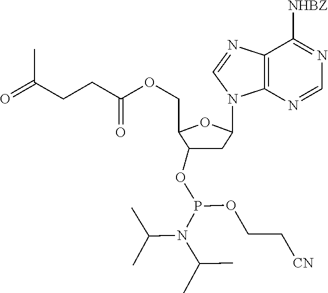 |

### Coupling

The pivotal phase of solid-phase oligonucleotide synthesis involves a coupling procedure that is integral to the automated and meticulously controlled process. This procedure entails the incremental attachment of a building block or successive sub building blocks to the developing chain of the oligonucleotide while it remains affixed to a stable support structure. Within the coupling phase, the oligonucleotide bound to the solid support, often connected to a controlled pore glass (CPG) bead or a similar substrate, may undergo initial deprotection. This deprotection unveils a chemically reactive moiety by removing the protective entities from the nucleotide building blocks integrated earlier in the process. Following deprotection, a nucleotide such as a phosphoramidite may be introduced into the reaction chamber. In some embodiments said phosphoramidites are nucleotides, either unmodified or modified, featuring a chemically reactive site at their 3′-end, poised for conjugation. In some embodiments the coupling reaction may transpire as the 3′-end of the incoming nucleotide phosphoramidite forms a phosphite triester linkage with the 5′-hydroxyl group of the immobilized oligonucleotide. This linkage creation is facilitated by a coupling agent, often composed of a phosphine activator and a hindered base. The coupling reaction progresses to completion under controlled chemical conditions, encompassing factors like reaction duration and solvent composition. A surplus of reagents is employed to ensure optimal yields and minimize the occurrence of incomplete coupling. Once the reaction concludes, any unreacted nucleotide phosphoramidite is thoroughly washed away, leaving behind the now-extended oligonucleotide securely bound to the solid support. This iterative sequence of deprotection, coupling, and washing may be repetitively undertaken to progressively construct the targeted oligonucleotide sequence. With each coupling step, the chain elongates by a building block or sub building block such as a singular nucleotide. The assembly of the sequence may unfold in a 3′ to 5′ direction, reflecting the inherent order of nucleotide addition observed in DNA and RNA synthesis.

Coupling according to the invention refers to the chemical attachment of a building block to an accessible reactive moiety comprising the solid support particle or intermediate. In some embodiments the first and/or second building block is coupled to the accessible reactive moiety by providing a building block comprising at least one accessible moiety and reacting said accessible building block moiety with the accessible reactive moiety of the solid support particle or intermediate. Reacting may refer to any chemical attachment of reactive moieties as known in the art.

While building blocks may be coupled as complete molecules (e.g., oligonucleotide sequence) in a single reaction according to the invention, in some preferred embodiments the building block is coupled to the accessible reactive moiety of the solid support particle by first reacting a sub-building block (e.g., nucleotide or oligonucleotide comprising said building block) comprising an accessible reactive moiety to the respective accessible moiety of the solid support particle or intermediate. In a further coupling step, a second sub-building block is attached to the previous sub building block. Said successive coupling of sub building blocks is performed until the complete build block according to the invention has been assembled. In some embodiments the building block according to the invention are oligonucleotides. In some embodiments said oligonucleotides are coupled to the respective reactive moiety of the solid support particle by successively coupling at least one nucleotide to said reactive moiety until the desired building block has been assembled.

For instance, if the building block is a sequence of 3 nucleotides AGC, coupling the respective building block according to the invention may refer to reacting the complete nucleotide or oligonucleotide comprising an accessible reactive moiety (building block) to the accessible reactive moiety of the solid support particle or intermediate in a single reaction using the designated group. In some embodiments said building block comprises the sub building blocks A, G, and C. Coupling said building block may refer to a multi-step reaction, wherein a first sub building block A comprising at least one accessible reactive moiety to the accessible reactive moiety of the solid support particle or intermediate thus generating a solid support sub building block intermediate using the designated group/chemistry. In a second reaction the next sub building block G is attached to the previous sub building block using the designated group. In a third step the third sub building block C is attached to the previous sub building block G using the designated group thus completing assembly of the complete building block and concluding the coupling step of the method according to the invention. In some embodiments, the designated group is removed from the coupled building block thus obtained before the first protective group, second protective group, or protective group x is coupled to the accessible moiety exposed by removing the designated group according to the invention.

Coupling is facilitated by utilizing the designated protective group according to the invention. Building blocks or sub building blocks are coupled to the respective reactive moieties by using the designated protective group chemistry. In some embodiments, the designated protected group is attached to the building block or sub building block before the coupling reaction. In some preferred embodiments, the designated protective group is selected from the group of DMTr, Fmoc or levulinyl. In a preferred embodiment, the designated protective group is DMTr.

### Combinatorial Chemistry

The method according to the present invention is particularly useful for the generation of complex oligonucleotide libraries using combinatorial chemistry approaches such as split-pool. Accordingly, in some embodiments, the steps (b) to (f) of the method of the invention are performed during successive rounds of split-pool synthesis.

In some embodiments step (a) further comprises segregating the solid support particles in j reaction vessels and performing steps (b) to (f) for each reaction vessel separately, wherein the building blocks used for each reaction in each reaction vessel may differ from the building blocks used in parallel reaction.

### Reaction Vessels

A reaction vessel refers to a vessel capable of containing solid phase synthesis particles, whether present as single particles of solid support particles or resins, or as multiple particles combined together in, e.g., a container. Non-limiting examples of reaction vessels are flasks, test tubes, wells of one or more microwell plates, columns or the like. In a preferred embodiment the reaction vessels are synthesis columns used for RNA or DNA synthesis known by the skilled person. In some embodiments, a reaction vessel refers to a column of an oligonucleotide synthesizer.

For the purposes of explanation, numerous specific details are set forth in the following description in order to provide a thorough understanding of the present invention. It will be apparent, however, to one skilled in the art that the present invention may be practiced without these specific details. In order that the invention may be readily understood and put into practical effect, particular preferred embodiments will now be described by way of the following non-limiting examples.

While the method according to the invention comprises at least steps (a) to (e), several preparations and optimization may be carried out before performing steps (a) to (e) of said method to maximize efficiency. Accordingly, an embodiment of the invention relates to a method comprising Step 1 to Step 12, which will be described in more detail below for illustration.

### Step (1): Designation of a Protection Group

Accordingly, the method of the present invention may start with Step (1) by designating a certain type of a protective group, which its usage should be maximized for the coupling reactions of the building blocks. This protective group shall be dubbed the designated group. Without loss of generality, this designated protective group can be selected on any single criterion or a combination of criteria that include chemical, environmental, commercial, and scientific reasons, such as the expected coupling efficiency, the orthogonality to the other protective groups, the costs of the monomer, and any other desired property. In some embodiments, the designated group is one of the following groups in Table 2.

TABLE 2
Protective groups and deprotecting agents

| Category | Protective group | Example of deprotection agent |
|---|---|---|
| Acid-labile | DMTr (or DMT) | 3% TCA |
| Base-labile | Fmoc | 5% piperidine |
| UV-labile | NPPOC | 6 Joule/cm² 365 nm light |
| | Bz-NPPOC | 2.7 Joule/cm² 365 nm light |
| | Sph-NPPOC | 0.5 Joule/cm² 365 nm light; or |
| | | 12 Joule/cm2 405 nm |
| Pd(0)-labile | Alloc | Pd(PPh₃)₄, dimedone |
| Fluoride-labile | TBDMS | 1M TBAF (Tetra-n- |
| | | butylammonium fluoride) |
| Hydrazine-labile | Levulinyl(Lev) | 0.4M Hydrazine, pyridine, acetic |
| | | acid |

In a preferred embodiment, the designated group is selected from the group of DMTr (Dimethoxy trityl), Levulinyl and Fmoc. In a more preferred embodiment, said designated protective group is DMTr.

DMTr is a protective group for hydroxy groups that is used primarily in nucleic acid chemistry. It is mainly used to protect the 5′ OH group of individual nucleotides in automated oligonucleotide synthesis. The protective group is acid labile and is usually deprotected with trifluoroacetic acid. Protection of the single nucleotide is done with the DMT chloride at room temperature in anhydrous pyridine, even in the presence of the 3′—OH group. DMT is so sterically demanding that the reaction occurs almost exclusively at the 5′-position (reactions on primary alcohols generally more preferred than on secondary alcohols).

In a more preferred embodiment, the DMTr designated group protects a phosphoramidite nucleoside. In the most preferred embodiment, the DMTr protects a 5′-phosphoramidite nucleoside.

### Step (2): Selection of a N-Set

In Step (2) an n-set may be selected. The n-set is a set of n protective groups, each of which exhibits orthogonality to the other n−1 protective group as well as orthogonality to the designated group, where n is the number of distinct synthesized oligos. The n-set may consist of any known set of chemically orthogonal protective groups. In a preferred embodiment, the n protective groups are selected from distinct categories of Table 2 above. In most preferred embodiment, n=2 and the n-set consist of Fmoc and Levulinyl.

### Step (3): Definition of One of the Protective Groups as Extra-Effort Group

Step (3) is optional. In this step, one of the protective groups in the n-set is defined as an extra-effort group. The extra-effort group is typically defined when the deprotection procedure is desired to be carried outside of a regular oligonucleotide synthesizer. In a preferred embodiment, when the n-orthogonal protective groups include a UV-labile group, this UV-labile group shall be defined as the extra-effort group.

### Step (4): Functionalization of Solid Support Particles

In Step (4) solid support particles are functionalized with a reactive moiety that is then protected by a mixture of n orthogonal protective groups. When an extra-effort group is not defined in Step (3), the mixture of protective groups is simply a mixture of the n-set. When an extra-effort group is defined, the mixture is all non-extra effort groups from the n-set. When an extra-effort group is defined, the mixture is all non-extra effort groups from the n-set plus the designated group. The solid support particles can be controlled porous glass (CPG), polystyrene based (e.g. toyopearl), or other types of resins. In a preferred embodiment, the solid support particles are CPG beads. In the most preferred embodiment, the CPG beads have pores of 1000 Å-3000 Å. Without loss of generality, the functional moiety upon deprotecting can be amino-terminated, hydroxyl-terminated, or carboxy-terminated groups. In some embodiments, the functionalized n-set protected particles are created by attaching an asymmetric with n-distinct protective groups, such as 1-[5-(4,4′-dimethoxytrityloxy) pentylamido]-3-[5-levulinyloxypentylamido]-propyl-2-[(2-cyanoethyl)-(N,N-diisopropyl)]-phosphoramidite for n=2 or Fmoc-protected, DMTr-protected dendrimer for n=2. In other embodiments, the particles are first functionalized with an unprotected active moiety, such as hydroxyl-terminated group, and then, a single round of addition of a mixture of n-distinct monomers, each of which with an orthogonal protective group is applied. Without loss of generality, this process can be carried out by:
(1) functionalizing the solid support particles with a linker, such as succinyl linker or a UV-labile linker such as the one described by Zhang & Taylor, 1999 (DOI: 10.1021/ja991300n)
(2) subjecting the particles to one round of coupling with 5′-protected nucleosides, where each nucleotide type has a distinct protective group.

### Step (5): Setting Synthesis Cycle Variable

According to the invention, in some embodiments the method comprises multiple synthesis cycles. Let c denote the protecting cycle. In Step (5), the process sets variable c to 0.

Step (6): Distribution of the Particles into Vessels

The method according to the invention may be combined with a combinatorial chemistry approach such as split-pool. Accordingly, in a Step (6), the solid support particles according to the invention may be distributed into j reaction vessels that are loaded to an oligonucleotide synthesizer, where j is a non-negative integer that denotes the number of distinct vessels for synthesis and is determined based on the synthesis plan. When j is bigger than 1, the process supports split-pool and j describes the number of sub-pools in the split step. In a preferred embodiment j>1. In a more preferred embodiment, j is an integer that is equal to 2^{x}3^{y}, where x is an integer with x≥2, and y is equal to 0 or 1. In the most preferred embodiment, j is an integer that is equal to 2^{x}3^{y}, where x is an integer with x≥5 and y=1. If an extra-effort group is defined, Step (6) occurs after the deprotecting Step (7). Each of Steps (7) to (11) are conducted for each vessel among the j-vessels.

### Step (7): Removal of Protective Group

In Step (7), one of the protective groups on the particles is selectively removed in order to expose a reactive moiety using methods known in the art for deprotecting thus revealing an accessible reactive moiety. The protective group to be removed is based on the following mutually exclusive conditions:
(a) if the designated protective group is on the particles, this is the protective group that is to be removed
(b) if c=0 and an extra-effort group appears on the particles, this group is to be removed
(c) if c=0 and one of the groups has an active moiety, Step (7) is skipped
(d) if none of the above, the process selects any protective group that is currently on the particles without any preferred order. In some embodiments, the deprotecting employs one of the methods in Table 2.
   By the end of this step, the process increments c by 1.

Following removal of the protective group a solid support particle comprising at least two reactive moieties, wherein one reactive moiety is accessible for chemical coupling of a building block and one moiety is blocked by a first protective group according to step (a) of the method according to the present invention is provided.

In Steps (8) to (10), the first building block is coupled to the accessible reactive moiety of the solid support particle.

Step (8): Washing with a Monomer

In Step (8), the reaction vessel is washed with a monomer that one of its moieties is protected with the designated protective group. In some embodiments, the monomer is a nucleoside whose either 3′ or 5′ are protected with the designated group. In a more preferred embodiment, the monomer is a nucleoside phosphoramidite, and in a further preferred embodiment, the monomer is a 5′-protected nucleoside phosphoramidite. In the most preferred embodiment, the monomer is a 5′-O-(4,4′-dimethoxytrityl)-R-3′-O—(N,N-diisopropylamino)phosphite, where R is either adenine, cytosine, guanine, thymidine, or uridine. Each of these monomers can include further modifications, which without loss of generality include 2′-F, 2′-O-Me, 5′-phosphorothiolate, 3′-phosphorothiolate, N1-methyl-pseudouridine. In some embodiments, the monomer further includes protective groups on its other functional moieties, such as exocyclic amines, 2′ alcohol, or phosphate. In some embodiments, the protective group is not reactive to the deprotecting conditions of any of the n other protective groups, such as acyl group or cyanoethyl.

### Step (9): Optional Synthesis Steps

Step (9) is again optional and includes one or more auxiliary oligonucleotide synthesis steps such as oxidation, sulphuration, or capping.

### Step (10): Deblocking of the Protective Group

In Step (10), the designated protective group is deblocked. In some embodiments, when the designated protective group is DMTr, Step (10) utilizes an acid wash. In more preferred embodiments, the acid is TCA. In the most preferred embodiment, the concentration of TCA is 3%.

Steps (8) to (10) are further repeated for k times, where k is a non-negative integer. The same designated protective group is used but the identity of the monomer of Step (8) can be changed in order to synthesize the desired k-long sequence of the oligonucleotide (building block).

### Step 11: Blocking of the Leading Moiety of the Growing Oligonucleotide

In Step (11), the leading moiety (either 5′ or 3′ in the case of oligonucleotide) of the growing oligonucleotide is blocked by a protective group (second protective group) that is subject to two conditions: (a) it is part of the n-set (b) it is not any protective group that is currently on the particles. In some embodiments, this protective group appears as a chloroformate ester. In some embodiments, this protective group is a protected nucleoside monomer whose chemical structure matches to the desired synthesis of the growing oligonucleotide. In this case, an optional Step (9) may be carried out. After Step (11), the solid support particles are protected by the n-set protective groups.

Steps (7) to (11) are repeated until the synthesis of the ki nucleotide of the i-th oligonucleotides is completed, where i runs from 2 to n. If c, the cycle counter is equal to n and no extra-effort group is defined, then Step (11) is optional and one of the oligonucleotides can have a leading moiety that is not protected.

### Step (12): Pooling of the Vessels

In Step (12) the j vessels are pooled together into j₀ pools, where j₀ is a non-negative integer such that j₀≤j. In a preferred embodiment, j₀=1, meaning that all j-vessels are pooled into one pool. Steps (5) to (12) are repeated until the synthesis of the desired library is completed.

### Advantages of the Invention

The process according to the present invention shows a plurality of advantages over the prior art. Due to the ability of the method to perform coupling reactions to assemble building blocks comprising nucleotides, when k=6, >85% of the synthesis is done utilizing the efficient chemistry of the designated protective group such as DMTr; 5′ DMTr deprotection is very well optimized and much more superior compared to other protective group leading to more efficient oligonucleotide synthesis. Many coupling chemistries using protective groups show poor coupling of G nucleotides but e.g., DMTr is robust in this regard. Thus, coupling other nucleotides is efficiently possible. Additionally, the method according to the invention allows utilization of block codes rather than a single letter.

### INDUSTRIAL APPLICATION

The method according to the invention has numerous industrial applications. For instance, said method is useful for the generation of a plurality of oligonucleotides in a single reaction vessel, the generation of complex libraries of linked oligonucleotides, wherein the chemical identity of one oligonucleotide is encoded by the other oligonucleotide generated during the synthesis according to the invention. Further useful applications, while not limiting, are the generation of complementary oligonucleotides and the use of the plurality of oligonucleotides for biological assays.

A further aspect of the present invention relates to a solid support particle, which comprises at least two reactive moieties useful for performing the method of the invention. Accordingly, a further aspect of the present invention refers to a compound according to formula (I)

$${( L 1 )}_{x} - T - {( L 2 )}_{y} \qquad formula ( I )$$

wherein
T denotes a solid support particle, or a solid support particle connected to L1 and L2 via a brancher or a doubler,
L1 denotes a spacer of formula (Ia):

$$- {( S 1 )}_{n} - {( B 1 )}_{m} - P 1 \qquad formula ( Ia )$$

L2 denotes a spacer of formula (Ib):

$$- {( S 2 )}_{o} - {( B 2 )}_{p} - {( P 2 )}_{q} \qquad formula ( Ib )$$

S1 and S2 independently from each other denote a linker,
B1 and B2 independently from each other denote a building block,
P1 denotes a protective group,
P2 denotes a second protective group orthogonal to P1,
n, m, o, and p independently from each other denote 0 or an integer of at least 1,
x, y independently from each other denote an integer of at least 1, and
q denotes either 0 or 1.

Surprisingly, it has been found that the compound according to formula (I) is useful as a solid support particle for the parallel synthesis of at least two distinct oligonucleotides according to the method of the present invention.

In some embodiments, T is a solid bead made from material selected from the group consisting of silica gel, glass, resins, metal, polymers, and polysaccharide. In some embodiments, T is selected from the group of controlled porous glass beads (CPG), polystyrene beads, and polyacrylamide beads. In a preferred embodiment, T is a CPG or a CPG covered with a doubler or brancher. In a further preferred embodiment, T is a CPG or a CPG covered with a doubler or brancher where the pore sizes are 1000 Å to 3000 Å.

In some embodiments, spacers S1 and S2 independently from each other represent linear or branched, saturated or unsaturated, aliphatic, cycloaliphatic, aromatic, optionally hydrocarbon radicals with 1 to 30 carbon atoms, said hydrocarbon radicals optionally interrupted by heteroatoms selected from oxygen, nitrogen, phosphor and/or sulphur and mixtures thereof. In some embodiments, S1 and S2 include a photolabile group.

In some embodiments, A1 and A2 independently from each other are acid groups selected from the group consisting of carboxylic acid, sulfuric acid, sulfonic acid, phosphoric acid, silicic acid, and mixtures thereof.

In some embodiments, B1 and B2 independently from each other are building blocks selected from the group of amino acids, peptides, nucleotides, and oligonucleotides. In a preferred embodiment B1 and B2 independently from each other are nucleotides or oligonucleotides. In a further preferred embodiment, B1 and/or B2 are DNA barcodes and encode information that can be read by high throughput sequencing.

In some embodiments, P1 is an acid-labile protective group and P2 is an orthogonal base-labile protective group to P1 or vice versa. In a preferred embodiment, P1 is DMTr and P2 is selected from the group of Fmoc, levulinyl, BSMoc, and NSMOC or vice versa. In a more preferred embodiment, P1 is DMTr and P2 is Fmoc or levulinyl or vice versa.

The compound according to the present invention is particularly useful for the synergistical generation of a plurality of synthetic oligonucleotides and barcode pairs according to the method of the present invention. In some embodiments, said barcode comprises DNA. In a further preferred embodiment, the barcode stores information that includes a code for error correcting or error detection based on methods known in the art.

The compound according to the invention is obtainable by methods known in the art. Reference is made for example to Rochambeau et al. (de Rochambeau D, Hirka S, Saliba D, Anderson S, Toader V, Dore M, et al. Orthogonal DNA barcodes enable the high-throughput screening of sequence-defined polymers. ChemRxiv 2022), Horn et al. (Horn T, Urdea MS. Forks and combs and DNA: the synthesis of branched oligodeoxyribonucleotides. Nucleic Acids Res. 1989 Sep. 12; 17 (17): 6959-67. Doi: 10.1093/nar/17.17.6959. PMID: 2780317; PMCID: PMC318426), U.S. Pat. No. 9,555,388B2, and EP1108783A2.

### BRIEF DESCRIPTION OF THE FIGURES

The most basic embodiment of the method according to the invention is exemplarily illustrated by the following scheme of FIG. 1 without limiting the invention to this embodiment. Depicted is a synthesis strategy for the generation of two chemically different oligonucleotides attached to the same soli support particle.

FIG. 2 depicts multiple possible embodiments of the coupling reaction(s) of the method of the present invention. Shown is schematically, the coupling of building blocks in a single reaction and protection of the reactive moiety generated using a protective group (A), the coupling of sub building blocks comprising the desired building block in separate reactions utilizing the designated group for each coupling step of the sub building blocks, followed by coupling of a protective group (here second protective group) to the solid support intermediate (B), and the coupling of sub building blocks, wherein the last sub building block is attached to the protective group (C).

The method according to the invention is further exemplarily illustrated for a certain embodiment by the flow scheme of FIG. 3 without limiting the invention to this embodiment. Depicted is a synthesis strategy for the generation of two different oligonucleotides attached to the same soli support particle using DMTr as designated group, X as first protective group, Y as second protective group, protective group x is the same as first protective group. Further depicted is the method of the invention performed during a split-pool synthesis approach.

FIG. 4 shows the yield as a function of the number of coupled nucleotides. The lower curve shows the results for a single chemistry with 92% coupling efficacy, the upper curve for the DMTr shuttle technology according to the present invention (99.5%); every 6nt and other chemistries with 80% efficacy.

FIG. 5 shows an alternative method according to the present invention, wherein X denotes a protective group, Y denotes an orthogonal protective group, rN denotes a ribonucleotide (rN-rN . . . are building blocks according to the present invention) and grey circles denote solid support particle.

FIG. 6A depicts the experimental design for optimization of Fmoc/Lev chemistry with DMTr chemistry. a) DNA synthesis by DMTr chemistry, b) addition of 5′ Fmoc or 5′Lev dC monomer denoted by “x”, c) Deprotection of 5′ Fmoc or 5′Lev protective group, d) DNA synthesis by DMTr chemistry with DMTr on, e) cleavage and deprotection of DNA oligo followed by HPLC analysis. FIG. 6B shows the results of experiment 1 as HPLC Chromatogram of single 5′ Fmoc monomer addition into DNA oligonucleotide followed by Lev deprotection and further DNA synthesis (nucleic acid sequences depicted are for exemplary purposes only).

FIG. 7 shows the results of experiment 2 as HPLC Chromatogram of single 5′ Lev monomer addition into DNA oligonucleotide followed by Lev deprotection and further DNA synthesis.

FIG. 8 shows experimental design for optimization of Fmoc/Lev chemistry with DMTr chemistry. a) DNA synthesis by DMTr chemistry, b) addition of 5′ Fmoc or 5′Lev dC monomer denoted by “x”, c) Deprotection of 5′ Fmoc or 5′Lev protective group, d) DNA synthesis by DMTr chemistry with final DMTr on, e) cleavage and deprotection of DNA oligo followed by HPLC analysis (nucleic acid sequences depicted are for exemplary purposes only).

FIG. 9 shows HPLC Chromatogram of three 5′ Fmoc monomer additions into DNA oligonucleotide followed by Fmoc deprotection and further DNA synthesis.

FIG. 10 shows HPLC Chromatogram of three 5′ Lev monomer additions into DNA oligonucleotide followed by Lev deprotection and further DNA synthesis.

FIG. 11 shows HPLC Chromatogram control DNA synthesized by standard DMTr chemistry.

FIG. 12 shows the experimental design of a complete run of an embodiment according to the method of the invention. a) DNA synthesis by DMTr chemistry, b) 1:1 mixture of C—X (5′ DMTr dC): C—Y (5′ Lev dC) monomers couplings, c) addition of C—Z (5′ Fmoc dC monomer), d) 5′ Lev deprotection, e) coupling of C—Y (5′ Lev dC monomer), f) 5′Fmoc deprotection, Z=UMI.

FIG. 13 shows a histogram indicating the number of individual sequencing reads (partially) mapping to the expected oligonucleotide sequence of the respective example, which were error free or showed indicated number of errors.

FIG. 14 Schematic of orthogonal synthesis of two oligonucleotide chains joined by an asymmetric doubler loaded with two orthogonal protective groups. a) Selective deprotection of the DMTr group of asymmetric linker b) synthesis of DNA oligonucleotide section Y by DMTr monomers with last addition of 5′-Fmoc-dT monomer, c) Lev deprotection of asymmetric doubler, d) top strand DMTr-dT synthesis with last addition of 5′-Lev-dC monomer, e) Fmoc deprotection from strand f) DNA synthesis by DMTr monomers g) deprotection and final oligonucleotide cleavage from solid support (nucleic acid sequences depicted are for exemplary purposes only).

FIG. 15 LC-MS trace of the purified oligonucleotide synthesised using Lev, Fmoc and DMTr chemistry. Calculated mass=22433 Da, observed=22432 Da.

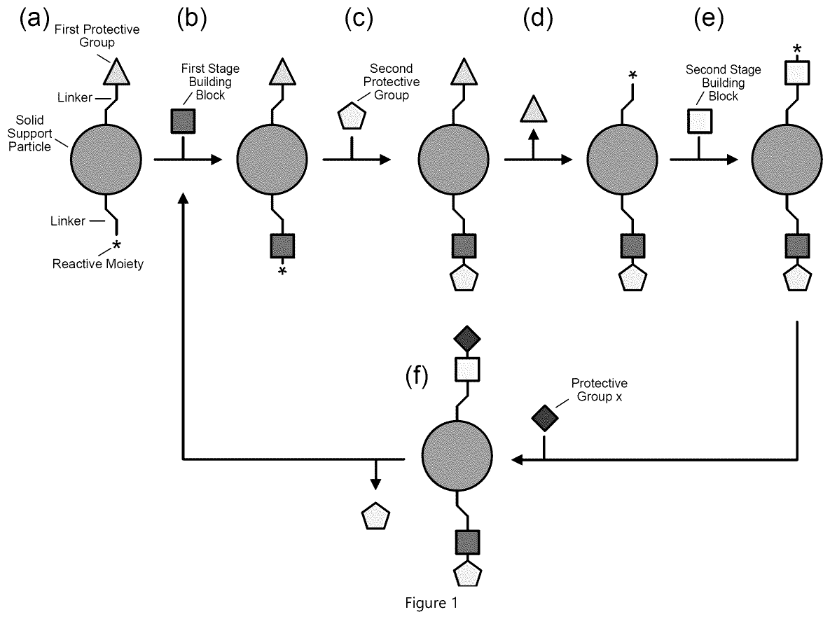

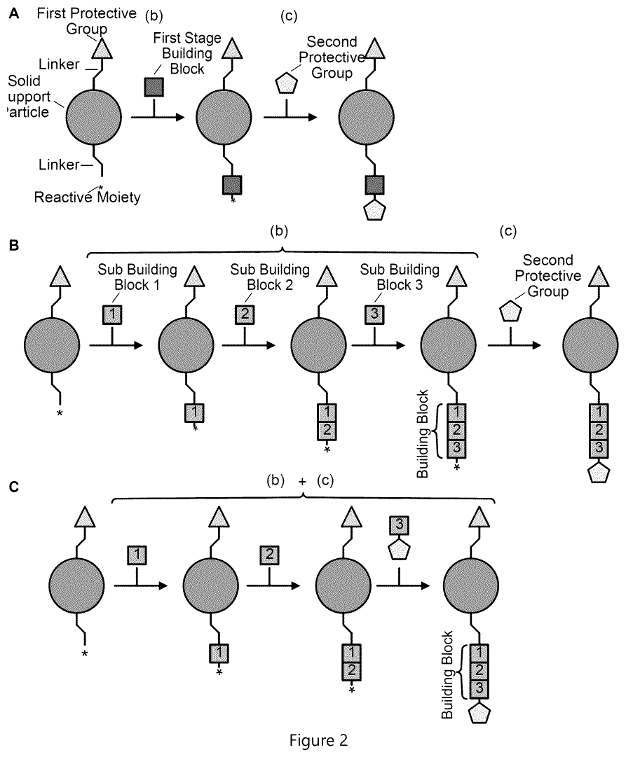

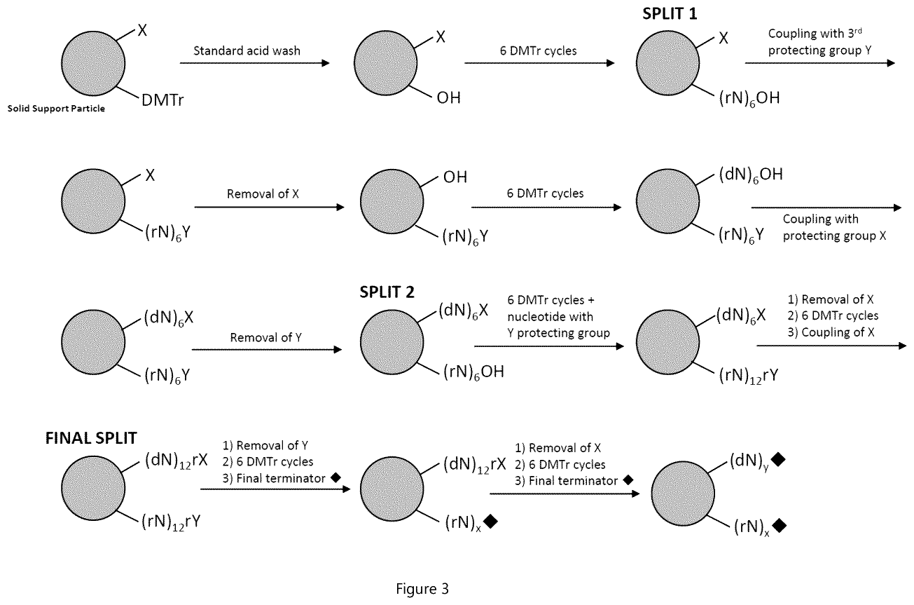

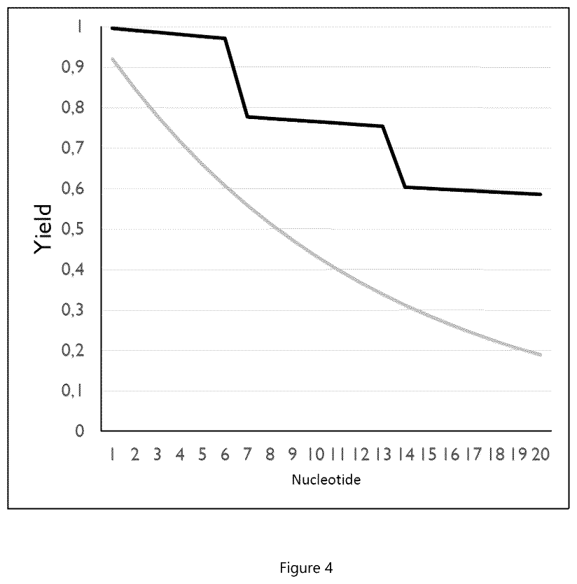

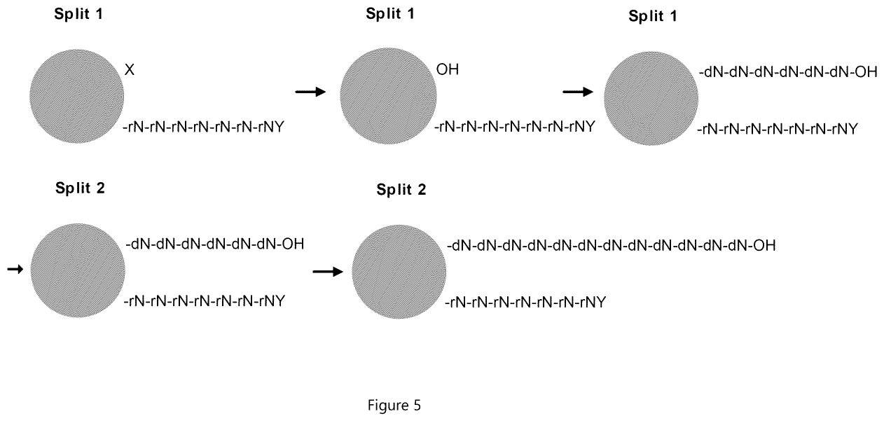

### EXAMPLES

### Example 1

### Selection and Optimization of Orthogonal Chemistries: Fmoc

The dimethoxytrityl (DMTr) protective group finds extensive application in safeguarding the 5′-hydroxy group in nucleosides, especially during oligonucleotide synthesis, such as in the solid-phase phosphite triester method. However, the specific requirements of the present invention require two more orthogonal protective groups that are compatible with solid-phase oligonucleotide synthesis. To meet this need, we have chosen to utilize i) Fluorenylmethoxycarbonyl (Fmoc) and ii) Levulinic (Lev) protective groups among the plethora of other protective groups listed in table 2 and known in the art.

Fmoc chemistry has found extensive use in solid-phase peptide synthesis as an amine protection group. It has been considered for potential use as an alcohol protection group during solid-phase oligonucleotide synthesis. In comparison to amines, alcohols are less nucleophilic, resulting in a weaker bond with the Fmoc group. This characteristic allows for milder deprotection conditions, making Fmoc deprotection orthogonal to DMTr chemistry.

Levulinyl esters are chemical compounds employed as alcohol protective groups in organic synthesis. They temporarily block the reactivity of alcohol functionalities in organic compounds, enabling selective chemical transformations without affecting the protected alcohol group. The Levulinyl group can be easily removed using hydrazinolysis, restoring the reactivity of the alcohol. This property offers greater control over reaction sequences and selective functionalization of specific alcohol groups, making it a valuable tool for oligonucleotide solid-phase synthesis. The Levulinyl group's stability and selective removal under mild conditions make it an attractive alternative to the standard DMTr chemistry. Notably, it is orthogonal to DMTr and Fmoc, remaining stable under basic conditions (unlike Fmoc) and unaffected by acidic conditions like DMTr. It can be selectively removed under almost neutral conditions using a pyridine: acetic acid buffer system in the presence of hydrazine, making it versatile for oligonucleotide synthesis.

Although the use of Fmoc-protected alcohols in oligonucleotide chemistry is not extensively explored, it shows promising potential as an alternative protective group with further optimization. The most effective method to remove the Fmoc protective group involves treating it with a solution of piperidine in DMF. Piperidine acts as a base with slight nucleophilic properties, allowing it to eliminate the Fmoc group by removing an acidic proton, followed by beta elimination of the 9-Methylene-fluorene group. The resulting fluorene group is highly reactive and forms stable adducts with nucleophiles irreversibly. Consequently, an excess of piperidine is necessary to quench the formed reactive byproduct and prevent undesired capping of the 5′ alcohol.

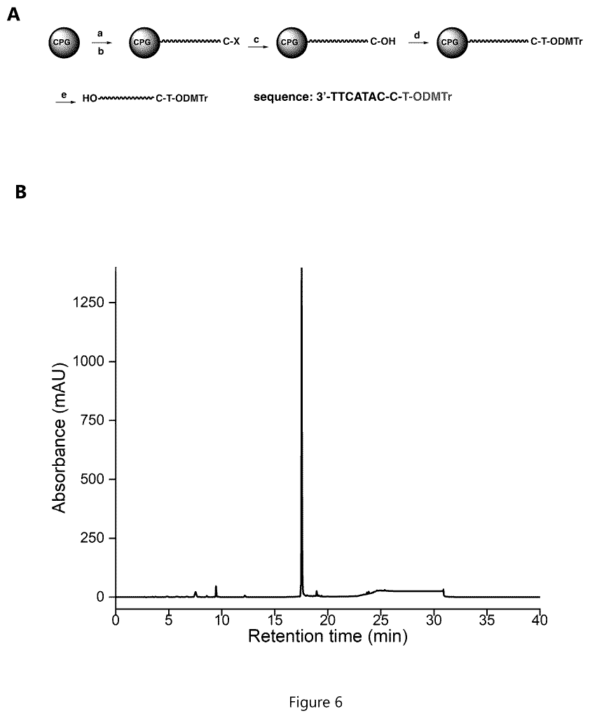

For this application, initially we optimized the Fmoc and Lev protective groups for singular DNA synthesis in combination with standard DMTr chemistry. In FIG. 6, we depicted the synthesis of a stretch of short DNA using DMTr chemistry, followed by the addition of either the 5′ Fmoc or Lev protected monomer. Subsequently, we deprotected the 5′ Fmoc or Lev protective group in a separate experiment, and DNA synthesis was terminated by adding the 5′ DMTr nucleotide, which acts as a lipophilic handle facilitating subsequent HPLC analysis. In these experiments, we synthesized a DNA of 9 nucleotides, wherein one nucleotide addition was either 5′ Fmoc or Lev protected while the others were 5′ DMTr protected.

In our investigation of Fmoc removal, we focused on two key factors: the concentration of piperidine and the reaction time. Through a series of screenings, we tested various piperidine concentrations, ranging from 1% to 10% (v/v), and deprotection times, ranging from 1 minute to 10 minutes. The results revealed that the most optimal conditions for deprotecting the Fmoc-protected alcohol were achieved using short reaction times of 30 seconds and a piperidine concentration of 4% in DMF. Under these conditions, we obtained an impressive yield of 97% (as shown in FIG. 6B).

Interestingly, we observed that longer deprotection times were unnecessary and even counterproductive, leading to the formation of unwanted byproducts during the reaction. We theorize that the basicity of piperidine triggers the premature removal of cyanoethyl-protective groups, which, in turn, leads to increased impurity formation through the branching of oligonucleotides. In summary, our findings suggest that employing a brief reaction time and a moderate piperidine concentration is crucial for achieving a high yield and minimizing unwanted side reactions during Fmoc removal.

### Example 2

### Selection and Optimization of Orthogonal Chemistries: Levulinyl

The levulinyl group was explored as a compatible alternative to DMTr and Fmoc protective groups for use in combination with the former two. However, during the initial experiment, few issues emerged. In some cases, when certain ratios of pyridine/acetic acid were used, pyridinium acetate precipitated in the presence of ACN, causing blockages in the DNA synthesizer tubing. As a result, these conditions proved to be unsuitable for the automated solid-phase synthesizer. To address this problem, the solubility of pyridinium acetate was found to be high in Dimethylformamide (DMF). Consequently, the automated synthesizer demonstrated compatibility with DMF, suggesting that it could be used as a co-solvent in the deprotection mixture. By incorporating DMF as a co-solvent, the issue of precipitation was resolved. However, it was observed that this modification slightly affected the reactivity of hydrazine.

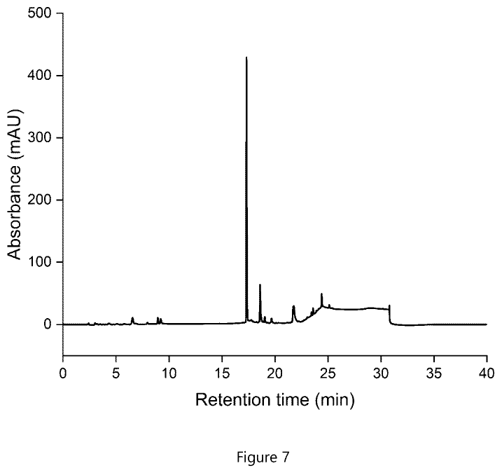

In the initial setup, the deprotection mixture contained 66% DMF. After making adjustments and testing, it was found that a composition of 50% DMF in the deprotection mixture resulted in no precipitation and maintained hydrazine reactivity within acceptable limits. Subsequently, a series of trials were conducted to optimize the 5′ levulinyl deprotection. This involved varying the percentages of hydrazine, pyridine, acetic acid, and deprotection time. Through careful experimentation, the most optimized condition was determined to be 0.4 M hydrazine, containing 30% pyridine, 20% acetic acid, in 50% DMF, with a deprotection time of 5 minutes. Under this optimized condition, a desired product yield of 96% was obtained (FIG. 7).

### Example 3

### Assessment of Orthogonality

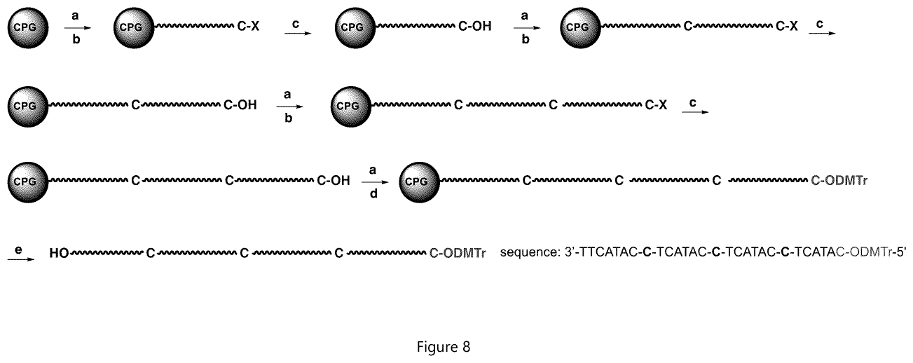

To further confirm the reliability of our initial findings, we designed a 28-nucleotide DNA sequence. In this sequence, three intermittent nucleotides used were protected with either 5′ Fmoc or Lev groups, while the rest were protected with 5′DMTr groups. The synthesis process is illustrated in FIG. 8. We used DMTr chemistry to synthesize a short stretch of DNA. Then, we introduced the 5′ Fmoc or Lev protected monomer to extend the sequence. Subsequently, we conducted a separate experiment to remove the 5′ Fmoc or Lev protective group, and then continued the DNA synthesis using DMTr chemistry. We repeated these steps until we achieved the desired DNA length, leaving the DMTr protective group intact on the last nucleotide to facilitate better HPLC analysis.

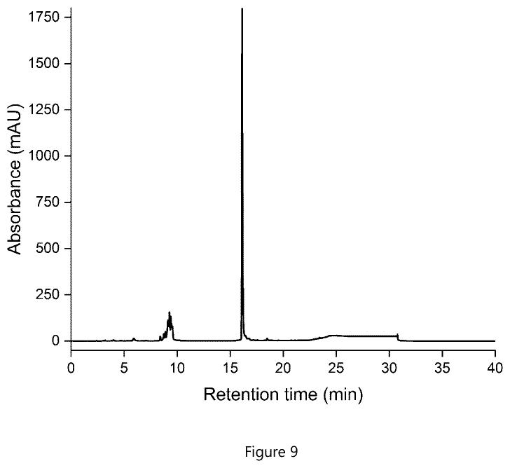

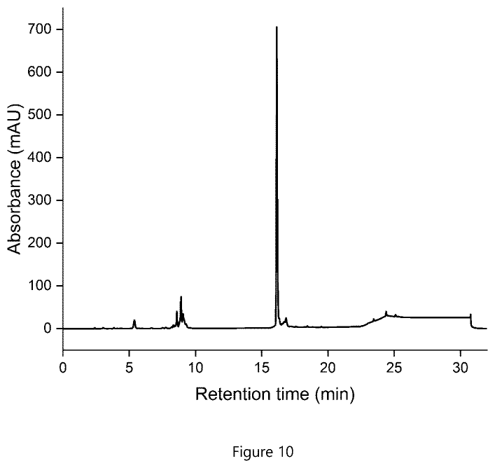

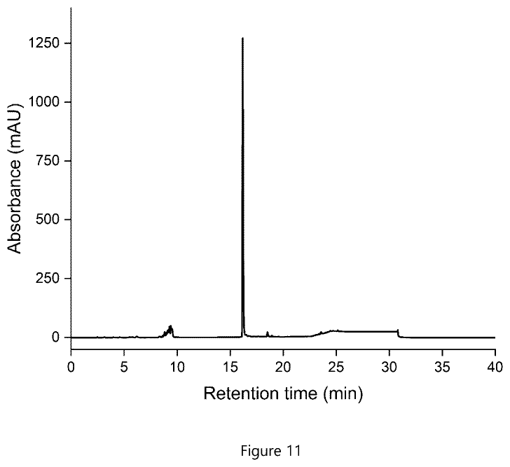

Using our finely optimized conditions, we successfully performed three rounds of 5′ Fmoc monomer additions, followed by their deprotection and DNA synthesis of seven nucleotides utilizing DMTr chemistry, resulting in a 71% yield of the desired product (FIG. 9). This is equal to an average incorporation efficiency of close to 98.8%. Similarly, when employing 5′ Lev monomers in this punctuated scenario, we achieved a 70% product formation (FIG. 10). This is equal to an average incorporation efficiency of 98.7%. As a point of comparison, our control experiment, where we conducted the entire DNA synthesis using standard DMTr chemistry, yielded 82% of the desired product (FIG. 11). This is equal to an average incorporation efficiency of 99.3%. These results suggest that the average incorporation efficiency of our simultaneous synthesis strategy reaches nearly (0.987/0.992=) 99% of the efficiency of standard DMTr. Above all, each of the three protective groups demonstrated exceptional orthogonality with one another.

### Example 4

### Designing an Experiment of Simultaneous Synthesis of Oligonucleotides

With these primary results we next implemented these protection groups in the parallel synthesis of two distinct oligonucleotides on a single bead (solid support). This experiment was designed such that it will synthesize two types of oligonucleotide molecules on the same bead, each of which was 70 nucleotides long.

DMTr was selected as the designated protective group and the n-set (n=2) consisted of Fmoc and Lev protective groups. We synthesized in parallel the payload of the oligonucleotides using a scheme of k=10, meaning that a burst of ten nucleotides of DMTr were used to create the payload in one oligo before protecting with either Fmoc or Lev and synthesizing the payload in the second oligo, creating unique sequences despite being at the same column. In total, we used this strategy to synthesize eight oligonucleotides in four columns, so each two sequences were synthesized in parallel using our strategy, thus setting j=4.

The payload of each oligonucleotide was a barcode. The eight barcodes were selected to have at least a minimal edit distance of 9. The barcodes were designed with “C” in selected payload positions (5′ to 3′): 1, 10, and 20. Table 3 specifies the payloads.

TABLE 3
Oligonucleotide barcodes

| | | Synthesizer | Protecting |
|---|---|---|---|
| # | Barcode | column | group |
| 1 | CCTTCGCAAC*TGCGTTACAC | 1 | Fmoc |
| | (SEQ ID NO: 1) | | |
| 2 | CACTCAGGAC*TCATCCGAAC | | Olev |
| | (SEQ ID NO: 2) | | |
| 3 | CGTGTGAGTC*CCGGAGTAAC | 3 | Fmoc |
| | (SEQ ID NO: 3) | | |
| 4 | CCCGGTTAAC*GACCAACAAC | | Olev |
| | (SEQ ID NO: 4) | | |
| 5 | CCGGAACTTC*AACTGTGGAC | 5 | Fmoc |
| | (SEQ ID NO: 5) | | |
| 6 | CGCCTTGTTC*CCTCTTCTAC | | Olev |
| | (SEQ ID NO: 6) | | |
| 7 | CCTAGAAGCC*GTTGGAACCC | 7 | Fmoc |
| | (SEQ ID NO: 7) | | |
| 8 | CAAGTGCCAC*ACAATAGCCC | | Olev |
| | (SEQ ID NO: 8) | | |

The * denotes the position that received one of the Fmoc or Lev n-set protective groups.
All sequences are presented 5′->3′

The payload of each oligo was designed to be flanked with identical forward and backward PCR annealing sites for subsequent DNA sequencing using DMTr synthesis. The left (5′-proximal) annealing site was designed to be: 5′-CACGACGCTCTTCCGATCT-3′ (SEQ ID NO: 9) and the right annealing site (3′-proximal) annealing site sequence to be: 5′-AGATCGGAAGAGCACACGTC-3′ (SEQ ID NO: 10).

Additionally, we designed each oligo to contain a 12-nt long Unique Molecular Identifier (UMI) using DMTr synthesis that will deploy all four bases in equimolar concentrations. The aim of the UMI was to quantify synthesis errors. It allows to enumerate distinct molecules with identical payloads and distinguish them from PCR duplicates.

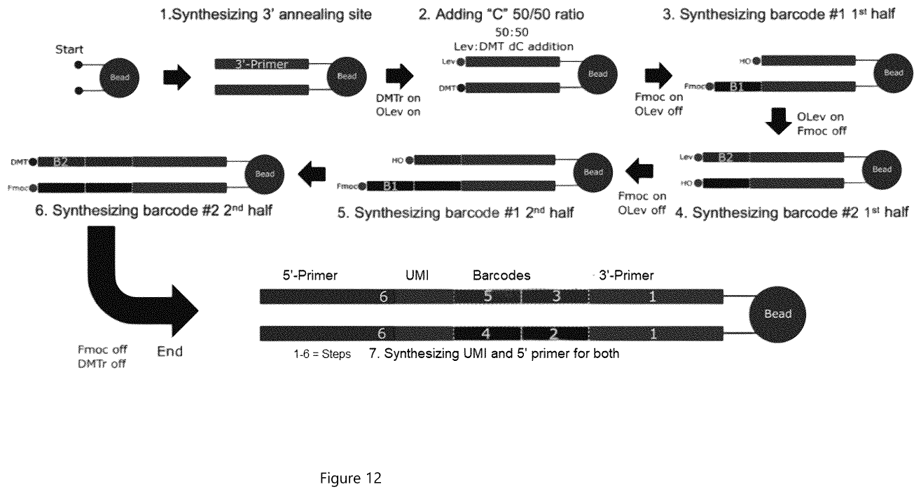

A schematic illustration of the synthesis design is depicted in FIG. 12.

### Example 5

### Simultaneous Synthesis of a Plurality of Oligonucleotides

We executed the synthesis of the design above. In this particular synthesis, we started a synthesis with controlled pore glass (CPG) beads with 1000 Å pores (Biosearch Technologies) that contained dC as a first nucleotide attached to beads.

We then synthesized 3′→5′ the 3′-proximal annealing site using standard DMTr chemistry to create the sequence: 5′-AGATCGGAAGAGCACACGTC-3′ (SEQ ID NO: 11).

Next, an equimolar concentration of 5′ DMTr and 5′ Lev dC (ChemGenes) monomers were coupled to this sequence which enabled the diversification of the sequence in the same column and even single bead. The use of 1:1 DMTr and Lev monomers allowed us to install half of the oligonucleotides in the beads with the DMTr monomer and the remaining half with the Lev monomer.

In each one of the four columns, we then incorporated 9 different nucleosides using 5′-DMTr protected nucleosides based on positions #19 to #11 of the sequences in the “Fmoc” rows in Table 3. For example, in synthesis column #1, we incorporated the following 5′-DMTr protected nucleosides: “A→C→A→T→T→G→C→G→T”. The next nucleotide incorporated into this sequence was a 5′-Fmoc dC monomer.

Afterwards, we selectively deprotected the Lev group using hydrazine in all four columns. We then incorporated 9 different nucleosides using 5′-DMTr protected nucleosides based on position #19 to #11 in the sequences of the “Lev” rows in Table 3. For example, in column #1, we incorporated the following 5′-DMTr protected nucleosides: “A→A→G→ (→C→T→A→C→T→T”. The next nucleotide incorporated into this sequence was a 5′-Lev dC monomer.

We selectively deprotected the Fmoc group in all four columns using 7.75% piperidine in N,N-Dimethylformamide. We then incorporated the next 9 different nucleosides using 5′-DMTr protected nucleosides based on position #9 to #2 in the sequences of the “Fmoc” rows in Table 3. For example, in column #1, we incorporated the following 5′-DMTr protected nucleosides: “A→A→C→G→C→T→T→C”. We then incorporated a nucleotide with a 5′-Fmoc dC monomer.

Next, the Lev group was deprotected in all four columns as described before. We then incorporated 9 different nucleosides using 5′-DMTr protected nucleosides based on position #9 to #2 in the sequences of the “Lev” rows in Table 3. For example, in column #1, we incorporated the following 5′-DMTr protected deoxy-nucleosides: “A→G→G→A→C→T→C→A. We then incorporated the 10th nucleotide with a 5′-DMTr dC monomer and was deprotected using standard DMTr deprotecting conditions.

Next, the 5′ Fmoc group from the top strand was removed using conditions as described above. This freed the 5′ hydroxyl group of both the strands in each column.

We then synthesize the UMI by 12 incorporations of an equimolar mixture of 5′-DMTr dA, dC, dG, and dT protected monomers.

Next, we synthesized the 5′-proximal annealing sequence: 5′-CACGACGCTCTTCCGATCT-3′ (SEQ ID NO: 9) by DMTr chemistry (nucleic acid sequences depicted are for exemplary purposes only).

Following the completion of chemical synthesis, the DNA oligonucleotides were cleaved and deprotected.

### Example 6

### Analysis of Simultaneous Synthesis

The previous examples clearly show the ability to simultaneously synthesize oligonucleotides according to the invention utilizing several protective groups.

Although each column contains two different sequences as these sequences are synthesized in pairs, the physico-chemical properties of these sequences are very similar hence their separation and analysis by HPLC or LC-MS is nearly impossible.

PCR-amplified the synthesized library using two primers: 5′-AATGATACGGCGACCACCGAGATCTACACTCTTTCCCTACACGACGCTCTTCCGATCT-3′ (SEQ ID NO: 12) and 5′-CAAGCAGAAGACGGCATACGAGATCGTGATGTGACTGGAGTTCAGACGTGTGCTCTTCCGATCT3′ (SEQ ID NO: 13). We then sequenced the library using Illumina iSeq, which was spiked with phi-X DNA to increase the sequencing complexity.

We removed phi-X and collapsed identical reads that had the same UMI and payload. Next, we tried to annotate each collapse reads to its original 8 possible payloads. This process revealed the following results in Table 4 (nucleic acid sequences depicted are for exemplary purposes only).

TABLE 4

| | | | | | % |
|---|---|---|---|---|---|
| | | | | | (collapsed |
| | | Synthe- | | # | reads |
| | | sizer | | (collapsed | without |
| # | Barcode | column | Chemistry | reads) | errors) |
| 1 | CCTTCGCAACTGCGTTACAC | 1 | Fmoc | 9,918 | 87.1% |
| | (SEQ ID NO: 1) | | | | |
| 2 | CACTCAGGACTCATCCGAAC | | Olev | 4,515 | 77.4% |
| | (SEQ ID NO: 2) | | | | |
| 3 | CGTGTGAGTCCCGGAGTAAC | 3 | Fmoc | 10,909 | 86.9% |
| | (SEQ ID NO: 3) | | | | |
| 4 | CCCGGTTAACGACCAACAAC | | Olev | 5,139 | 66.2% |
| | (SEQ ID NO: 4) | | | | |
| 5 | CCGGAACTTCAACTGTGGAC | 5 | Fmoc | 10,740 | 91.1% |
| | (SEQ ID NO: 5) | | | | |
| 6 | CGCCTTGTTCCCTCTTCTAC | | Olev | 14,464 | 74.0% |
| | (SEQ ID NO: 6) | | | | |
| 7 | CCTAGAAGCCGTTGGAACCC | 7 | Fmoc | 7,323 | 90.1% |
| | (SEQ ID NO: 7) | | | | |
| 8 | CAAGTGCCACACAATAGCCC | | Olev | 4,963 | 72.3% |
| | (SEQ ID NO: 8) | | | | |

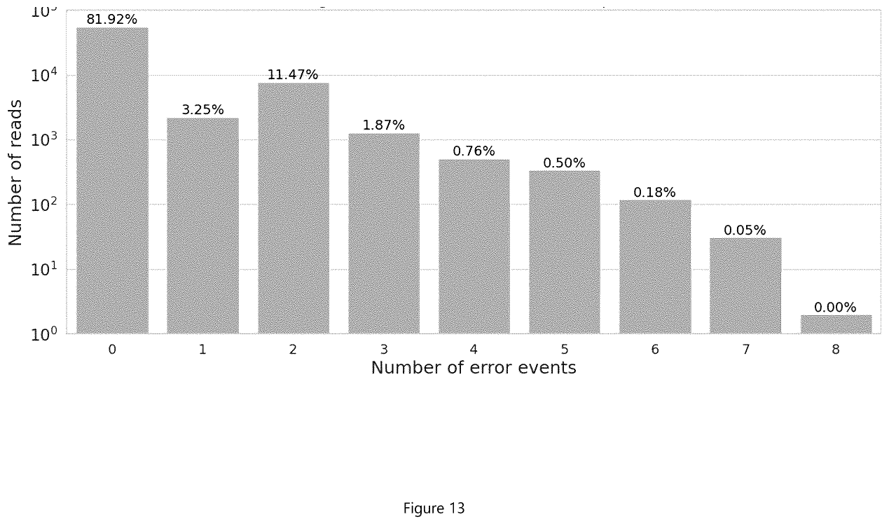

The sequencing results revealed that the DMTr, FMOC and Levulinyl chemistries exhibited an exceptional degree of orthogonality. Within each pair of oligonucleotides, the top strand exhibited a slightly higher percentage of error-free reads. Nevertheless, approximately 82% of the reads demonstrated an absence of any errors, a fact corroborated by the histogram depicting the distribution of error events per read (FIG. 13). Moreover, the synthesis error rate was comparable to that achieved by the DNA Fountain technique as per the capabilities demonstrated by Erlich et al. (Y. Erlich and D. Zielinski, Science, 2017, 355, 950-954).

We also quantified cases where chimeric payloads were created due to protection failures of Fmoc or Lev. The results found that on average only 0.5%, 0.24%, and 0.07% of the reads were of chimeric configurations stemming from failures of Lev capping after synthesizing the first half of the payload, failures of the last Fmoc capping, and failures of first Fmoc capping respectively.

The results presented above clearly demonstrate the usefulness of the method in generation of a plurality of oligonucleotides maximizing the coupling efficiency of a designated protective group (here DMTr), while taking advantage of a first and second protective group (here Fmoc and Lev), which are orthogonal to each other. Simultaneous synthesis of two different DNA oligonucleotides on a single support was not only possible but also time, cost, and yield efficient.

### Example 7

### Simultaneous Synthesis on Solid Support Beads with an Asymmetric Doubler

In this example, we present the procedure for simultaneously synthesizing two distinct oligonucleotide branches interconnected starting with an asymmetric doubler (Lev) (1-[5-(4,4′-dimethoxytrityloxy) pentylamido]-3-[5-levulinyloxypentylamido]-propyl-2-[(2-cyanoethyl)-(N,N-diisopropyl)]-phosphoramidite) (Glen Research) that is attached via linkers to CPG beads. The designated protective group was DMTr and the n-set (n=2) was Fmoc and Lev. This experiment effectively showcases the practicality of introducing multiple oligonucleotide synthesis within a singular oligonucleotide strand. Furthermore, it establishes the adaptability of our innovation to diverse building blocks beyond nucleotides, including but not limited to Biotin, PC-spacer, puromycin, PEG spacer-9, and PEG spacer-18.

Our solid support in this experiment was CPG beads with 2000 Å pores that are attached to a puromycin aminonucleoside (Chemgenes).

We then used standard DMTr synthesis to add three dT phosphoramidite monomers, followed by the spacer-9 phosphoramidite (9-O-Dimethoxytrityl-triethylene glycol, 1-[(2-cyanoethyl)-(N,N-diisopropyl)]-phosphoramidite, by Biosearch technologies) and another eight DMTr-protected dT monomers.

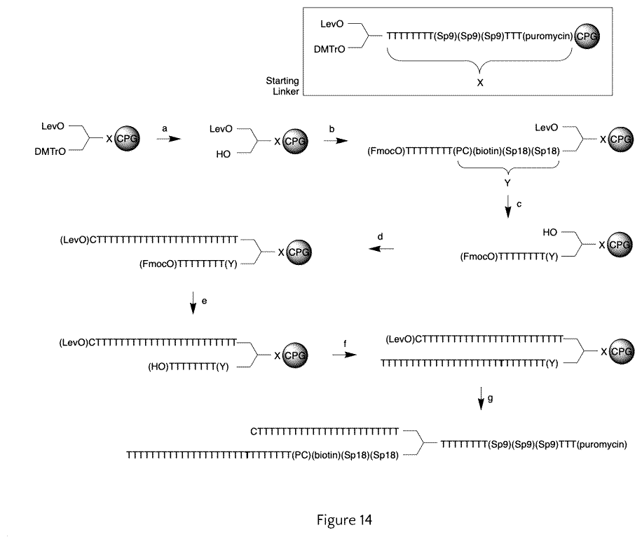

Next, we incorporated the asymmetric doubler, which contains two orthogonal protective groups DMTr and Lev (FIG. 14). This step introduces the branching of the DNA allowing orthogonal synthesis of two paralleled oligonucleotides.

We then continued the synthesis using DMTr monomers in order to extend one of the oligonucleotides. We will term this oligonucleotide the “bottom strand” and the other oligonucleotide which will be extended from the Lev-protected will be termed the “top strand”.

First, we added two spacer-18 monomers (18-O-Dimethoxytritylhexaethyleneglycol, 1-[(2-cyanoethyl)-(N,N-diisopropyl)]-phosphoramidite, Biosearch technologies) to the bottom strand followed by biotin TEG (1-Dimethoxytrityloxy-3-O—(N-biotinyl-3-aminopropyl)-triethyleneglycolyl-glyceryl-2-O-(2-cyanoethyl)-(N,N-diisopropyl)-phosphoramidite, Biosearch technologies) and a PC-photocleavable linker (3-(4,4′-Dimethoxytrityl)-1-(2-nitrophenyl)-propan-1-yl-[(2-cyanoethyl)-(N,N-diisopropyl)]-phosphoramidite, Biosearch Technologies). All of these used standard DMTr-protected building blocks and phosphoramide chemistries. These steps were followed by the addition of seven DMTr-dT monomers. Finally, we added an Fmoc-protected dT monomer.

Next, we deprotected the Levulinyl group from the other end of the asymmetric doubler to release a reactive hydroxyl group for oligonucleotide synthesis of the top strand. We added 24 DMTr-dT monomers, which selectively extended the top strand. Finally, we added a Lev-dC monomer.

Next, we deprotected the Fmoc group from the bottom strand to the 5′-hydroxyl which is now available for synthesis. This is followed by addition of 20 DMTr-dT monomers (FIG. 14).

Finally, we cleaved the doubled construct from the bead so that the puromycin was on the 3′ distant end and deprotected it using aqueous ammonia for five hours at 55 degrees Celsius, which in parallel also removes the 5′ Lev protective group from the top strand. The oligonucleotide was then purified by denaturing PAGE.

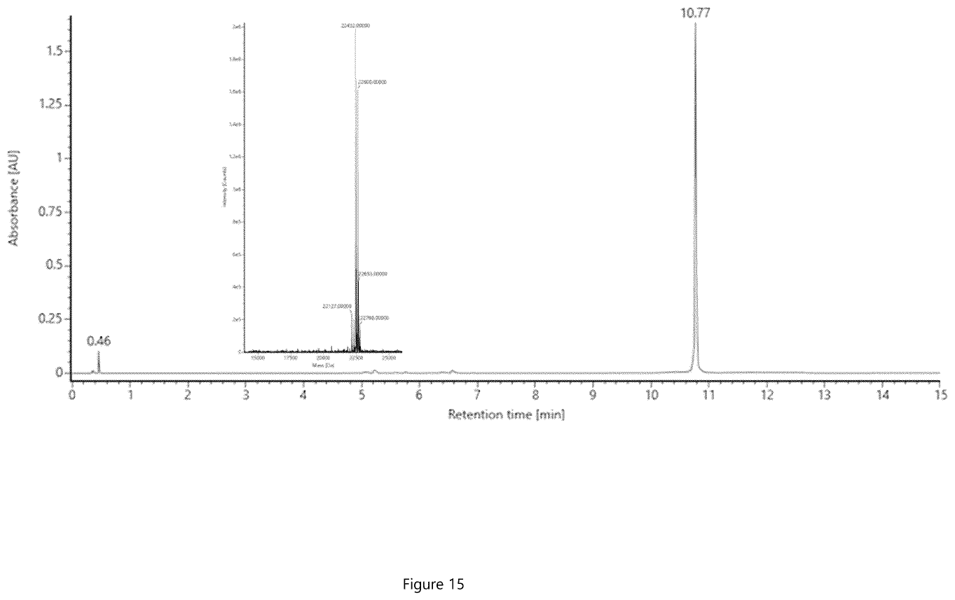

The purified construct was analyzed by LCMS. This established the molecular integrity of the construct obtained using our simultaneous synthesis (FIG. 15). The calculated mass was 22433 Daltons and the observed mass was indeed 22432 Daltons.

This example further demonstrates that the Lev, Fmoc, and DMTr protective groups have excellent orthogonal properties. It shows that our strategy can work with different type of solid support, including asymmetric doublers, and that our simultaneous synthesis strategy can work with various chemical elements beyond the standard deoxynucleotides nucleotides to create complex molecular structures.

Nucleic acid sequences depicted in the figures and examples are purely exemplary to illustrate the embodiments of the invention in regards of sequence length and the likes. The skilled person in the art appreciates that said sequences are by no means limiting nor do the depicted sequences of bases disclose information relevant for the understanding or execution of the present invention.

In the following section, general procedures and experimental settings are concisely depicted.

### General Information and Instrumentation

All starting materials were obtained from commercial suppliers and used without further purification. Commercially available DNA monomers are acquired from either Biosearch Technologies, Glen Research or ChemGenes. DNA monomers containing 5′ Fomc and Lev protective groups were obtained from ChemGenes. All the solid support and reagents for oligonucleotide synthesis were purchased from either Biosearch Technologies or Sigma Aldrich. Standard automated solid-phase synthesis was performed on a K & A H-8 SE synthesizer. HPLC purification was carried out on Dionex UltiMate 3000. DNA oligomers quantification measurements were performed by UV absorbance with NanoDrop Lite spectrophotometer from Thermo Scientific.

### General Solid-Phase Synthesis Procedure

Standard DNA synthesis was performed on a 1 μmol scale, starting from 1000 Å LCAA-CPG solid support. Amidites were dissolved in dry acetonitrile to obtain 0.1 M solutions. Removal of the 5′ DMT protective group was carried out using 3% dichloroacetic acid in dichloromethane. For the removal of 5′ Fmoc group, 4% piperidine in DMF is used while the removal of 5′ Lev group was carried out by 0.4 M hydrazine, containing 30% pyridine, 20% acetic acid, in 50% DMF. For standard DNA amidites 1 min coupling time was used while for 5′ Fmoc and Lev amidites an extended coupling time of 3 minutes was used.

### General Procedure for RP-HPLC Analysis

HPLC analysis was performed on Dionex UltiMate 3000 System. Column: Kinetex 5 um EVO C18 250 mm*4.6 mm, CV=4.15 mL. Solvents A: 0.1 M TEAB Buffer, pH 7.2, B: ACN. Following gradient in table 5 was used for the elution of the sample.

TABLE 5

| Retention (min) | % A | % B |
|---|---|---|
| 0 | 89.5 | 10.5 |
| 4 | 89.5 | 10.5 |
| 12 | 77 | 23 |
| 20 | 30 | 70 |
| 20.1 | 5 | 95 |
| 28 | 5 | 95 |
| 28.1 | 89.5 | 10.5 |
| 32 | 89.5 | 10.5 |

For each analytical separation approximately 1 nmol of crude oligomers was injected in 50 μL of millipore water. Detection was carried out using a diode-array detector, monitoring absorbance at 260 nm.

### General Procedure for LCMS Analysis

LCMS analysis was performed on a Waters BioAccord Time of flight RDa and UPLC system. Column: Acquity Premier Oligonucleotide BEH C18, 130 Å, 1.7 μM, 2.1×50 mm (PN:186009484). Solvent A: 80 mM 1,1,1,3,3,3-Hexafluoroisopropanol (HFIP, Apollo), 7 mM triethylamine (Sigma) in water, B: 40 mM HFIP, 3.5 mM triethylamine in 1:1 methanol (fisher scientific UPLC grade) and water. The gradient for analysis is detailed in table 6.

For each analysis 10 μL of oligonucleotide was injected at a concentration of 10 μM in deionized water. UPLC analysis was monitored at 260 nm and total ion count (TIC) of eluted peaks was deconvoluted using Waters connect software package.

TABLE 6

| Retention (min) | % A | % B |
|---|---|---|
| 0 | 78 | 22 |
| 10 | 60 | 40 |
| 11 | 15 | 85 |
| 12 | 15 | 85 |
| 12.1 | 78 | 22 |
| 15 | 78 | 22 |

### Filtration and Library Multiplexing

After Solid phase synthesis and deprotection each sample was dissolved in 1 ml water and filtered through a 0.22 μm Spin-X column (Costar-PN21121000). Sample concentration was measured using a nanodrop 2000 by absorbance at 260 nm. Samples were diluted to 80 ng/μl in water and 20 μl of each sample was combined to create the encoded library. The library concentration was diluted to 10 ng/μl in water.

### PCR and PAGE Purification

1 μL (10 μM) forward and reverse primer, 10 μL 2× Phusion master mix (NEB-M0530S) and 7 μL water was combined with 1 μL (10 ng) of library template for library preparation. PCR details: step 1-98° C., 30 s, step 2-98° C., 10 s, step 3-60° C., 10 s, step 4-72° C., 10 s, back to step 2 for 15 cycles, step 5-72° C., 60 s, step 6-4° C., hold. 1 μL of the sample was taken and analyzed by Agilent D1000 tape screen. PCR product was purified using a 10% denaturing urea PAGE at 200 V for 1 h. The amplified DNA library was cut from the gel and extracted in 1 ml of water for 16 h at rt. DNA was desalted using an Amicon Ultra 0.5 mL 3000 MWCO (Merck—UFC500324) and washed with 4×0.5 ml water.

qPCR Library Quantification

Template library was diluted to 20 nM in a suitable qPCR buffer. From this a serial dilution was prepared of 1:1000, 1:10,000, 1:100,000, 1:1,000,000 in qPCR buffer. 6 μL of Kapa master mix (Roche-KK4873-07960336001) was combined with 4 μL of each sample and run in duplicate. qPCR was performed using a Quantstudio 3 (applied biosystems, reagents mode—SYBR green, run mode-standard, experiment type-standard curve), PCR details: step 1-95° C., 300 s, step 2-95° C., 30 s, step 3-60° C., 45 s, back to step 2 for 35 cycles. Based on qPCR analysis the concentration of the library was determined to be 44 nM.

### Sequencing

iSeq 100 i1 Reagent v2 (Ilumina 20031371) was thawed overnight at rt. Stock DNA library (44 nM) was diluted to 1 nM in sequencing buffer (10 mM Tris-HCl PH 8.5). 10 nM PhiX control v3 (Ilumina FC-110-3001) was diluted to 1 nM in sequencing buffer. To 90 μl of sequencing buffer was added 4 μL of 1 nM PhiX and 6 μL of 1 nM DNA library, final concentration 0.1 nM consisting of 40% PhiX and 60% DNA library. 20 μl of sample was pipetted into the flow cell and the cartridge was placed into the iSeq 100 (Ilumina). Read length was set to 26 bp and run time was 6-7 hrs.

1. A method for simultaneous synthesis of a plurality of oligonucleotides, comprising or consisting of:
(a) providing solid support particles comprising at least two reactive moieties, wherein one reactive moiety is accessible for chemical coupling of a building block and one moiety is blocked by a first protective group, a first stage building block and a second stage building block;
(b) coupling said first stage building block to said accessible reactive moiety generating a solid support intermediate;
(c) coupling a second protective group to said coupled first stage building block of the solid support intermediate thus obtained;
(d) removing the first protective group of the solid support intermediate thus obtained allowing access to the reactive moiety;
(e) coupling a second building block to said accessible reactive moiety thus obtained;
(f) optionally coupling the reactive moiety of the support intermediate thus obtained with protective group x, removing said second protective group, and repeating (b) to (e) for n times, wherein the first and second building block can differ from the building blocks provided in previous cycle;
wherein n denotes an integer number of at least 1,
the second protective group is orthogonal to the first protective group and protective group x is orthogonal to the second protective group.

2. The method of claim 1, wherein the first and/or second building block comprises or consists of a designated protective group.

3. The method of claim 2, wherein the first protective group, second protective group, and the designated protective group are chemically orthogonal to each other.

4. The method of claim 1, wherein (b) and (e) comprise at least one reaction cycle comprising coupling and deprotection of the building blocks, generating a solid support intermediate.

5. The method of claim 4, wherein the first and/or second building block is assembled by coupling sub building blocks to a respective accessible moiety using the designated group until a chemical structure of a complete building block has been assembled.

6. The method of claim 1, wherein said protective groups are independently selected from Table 2.

7. The method of claim 5, wherein said designated protective group is selected from the group of DMTr Levulinyl and Fmoc.

8. The method of claim 7, wherein said designated protective group is DMTr.

9. The method of claim 5, wherein the first protective group and second protective group are either Levulinyl or Fmoc, and the first protective group and the second protective group are different from each other.

10. The method of claim 1, wherein the protective group x is the same as the first protective group.

11. The method of claim 5, wherein the building blocks and/or sub-building blocks utilize phosphoramidite chemistry.

12. The method of claim 5, wherein said building block and/or sub-building block is selected from the group of nucleotides, oligonucleotides, nucleosides and/or deoxynucleosides.

13. The method of claim 1, wherein said building block is selected from table 1.

14. The method of claim 1, further comprising (g) releasing a plurality of oligonucleotides thus obtained from a solid support particle.

15. The method of claim 1, wherein at least one oligonucleotide of said plurality of oligonucleotides comprises a barcode allowing the identification of chemical identity of other oligonucleotides coupled to a same solid support particle.

16. The method of claim 1, wherein (b) to (f) are performed during at least one round of split-pool synthesis.

17. The method of claim 1, wherein the solid support unit is selected from the group of glass, poly(acryloylmorpholine), silica, controlled pore glass (CPG), polystyrene, polystyrene/latex, teflon, acrylate, polyacrylamide, agar, agarose, chemically modified agars and agaroses, carboxyl modified teflon, nylon and nitrocellulose.

18. The method of claim 17, wherein said solid support further comprises an asymmetric doubler.

19. A compound according to formula (I) 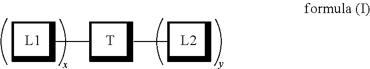
wherein
T denotes a solid support particle, or a solid support particle connected to an asymmetric doubler or brancher,
L1 denotes a spacer of formula (Ia): 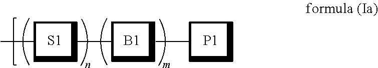
L2 denotes a spacer of formula (Ib): 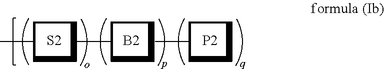
S1 and S2 independently from each other denote a linker,
B1 and B2 independently from each other denote a building block,
P1 denotes a protective group,
P2 denotes a second protective group orthogonal to P1,
n, m, o, and p independently from each other denote 0 or an integer of at least 1,
x, y independently from each other denote an integer of at least 1, and
q denotes either 0 or 1.

20. The compound of claim 19, wherein P1 and P2 independently from each other are Fmoc or DMTr, while P1 and P2 are different from each other.

21. The compound of claim 20, wherein P1 and P2 independently from each other are Fmoc or Lev, while P1 and P2 are different from each other.

22. The compound of claim 21, wherein P1 and P2 independently from each other are Lev or DMTr, while P1 and P2 are different from each other.

23. The compound of claim 19, wherein T is a controlled pore glass bead (CPG).

24. The compound of claim 23, where the CPG bead has pores of the size 1000 Å to 3000 Å

25. The compound of claim 24, where the CPG beads are covered with an asymmetric doubler and/or brancher.

26. The compound of claim 25, where doubler and/or brancher is 1-[5-(4,4′-dimethoxytrityloxy) pentylamido]-3-[5-levulinyloxypentylamido]-propyl-2-[(2-cyanoethyl)(N,N-diisopropyl)].
